(19) United States
(12) Patent Application Publication
(10) Pub. No.: US 2012/0109286 A1
(43) Pub. Date: May 3, 2012

(54) ANTIMICROBIAL COATINGS

(76) Inventors: Andrew Johnson, Bath (GB); Neil Poulter, Bath (GB); Andrew Jenkins, Bath (GB)

(21) Appl. No.: 13/145,431

(22) PCT Filed: Jan. 20, 2010
(86) PCT No.: PCT/GB2010/000089

§ 371 Date: Nov. 29, 2011

(30) Foreign Application Priority Data
Jan. 20, 2009 (GB) 0900961.4
Mar. 3, 2009 (GB) 0903658.3

(51) Int. Cl. A61F 2/06; B01D 39/00; B05D 3/00

(52) U.S. Cl. 623/1.46; 427/2.1; 55/524; 210/506

(57) ABSTRACT

The present invention relates to methods for forming antimicrobial coatings, and to compounds suitable for forming the antimicrobial coatings, and to substrates coated with the antimicrobial coatings. The coatings are formed by vapour phase plasma polymerisation of metal ion complexes each having at least one polymerisable ligand, which includes a polymerisable moiety such as a C—C double bond or a C—C triple bond.

### FIELD OF THE INVENTION

The present invention relates to materials and methods for forming antimicrobial coatings on substrates by plasma deposition, and in particular to methods which employ metal ion complexes.

### BACKGROUND OF THE INVENTION

The discovery of penicillin in the 1928 and other antibiotics subsequently, led to a wide spread belief that the fight against microbial infection had effectively been won. Unfortunately, in recent decades the evolution of resistant strains of bacteria has become a growing problem, with the emergence of so called ‘hospital super-bugs’ including Methicillin Resistant Staphylococcus aureus (MRSA).¹ Many bacteria that are not particularly antibiotic resistant in their free swimming or planktonic phase can become resistant when they colonize surfaces, either abiotic such as IV lines and urinary catheters, or biotic such as lungs and soft tissue. A good example of such a bacteria is Pseudomonas aeruginosa, which can be lethal to immuno-compromised patients due to its ability to form complex colonies and biofilms.² A biofilm is a structured, viscoelastic community of bacterial cells enclosed in a self-produced polymeric matrix adhered to an inert or living surface at a solid-liquid interface.³ Such sessile communities are very hardy and have been proven to withstand extreme antibiotic treatment (up to 1000 times more than planktonic cells).⁴ Other problems associated with microbial activity, particularly on surfaces, include those associated with microbial action on the skin and mucous membranes, for example skin and wound infections, and dermatitis.

There is therefore a clinical need to create coatings that can be applied to surfaces of substrates including those used in medical applications, such as medical devices and medical plastics, and substrates which are in contact with the skin during normal use, for example nappies and wound dressings, that can either resist initial microbial attachment and/or kill microbes that do attach onto such surfaces. In principal bacteria can be repelled from surfaces by using ultrahydrophobic surfaces or creating surfaces that are sterically unfavourable for bacteria such as polyethylene oxide and polyethylene glycol coatings.^{5 6 7} These surfaces are effective over short periods, but tend to lose effectiveness with time.

Other antimicrobial strategies are designed to kill bacteria that do attach. A number of approaches are documented in the literature, including impregnating surfaces with agents such as Triclosan™ (5-chloro-2-(2,4-dichlorophenoxy)phenol). However there are concerns about the long term effects of releasing Triclosan into the natural environment and long term retention in humans.⁸ A recently reported approach to killing S. aureus used toluidine blue O-tiopronin-gold nanoparticle conjugate, which became effective on irradiation with white light or red laser light.⁹ Other approaches include using antimicrobial cationic polymers and antimicrobial peptides. Advances in this area have been reviewed by Gabriel et al.¹⁰

However, the most commonly investigated approach to designing bactericidal properties into coatings has been to utilize the bacterial toxicity of silver, particularly Ag (I). Silver has long been known to have antimicrobial properties, due to its ability to inactivate enzymes within the bacteria required for DNA replication. Silver is attractive as an antimicrobial agent as, unlike some antibiotics and halogens, it does not display toxicity or carcinogenicity to human cells in low concentrations.¹¹ Silver (I) cations (Ag⁺) are believed to act by binding strongly to electron donor groups containing sulphur, oxygen or nitrogen, which are plentiful in bacterial cells and replacing essential metal ions such as Ca²⁺ and Zn²⁺.¹² Ag⁺ ions have been shown to deactivate enzymes required for DNA replication by blocking the helix from unwinding,¹³ and may also perform activity at cell membranes, where it reacts with thiol (—SH) groups, causing damage to the microbe and activating self-destruct genes.¹⁴

Plasma deposition is becoming established as an effective method for modifying surfaces, including metal and plastic surfaces, with thin organic functional films. The method involves introducing monomeric material as a vapour at low pressure and applying radio frequency power, usually via external electrodes and an impedance matching unit. The plasma contains a mixture of radicals, electrons and photons. The precise physical processes that take place in the plasma are complex, but it is generally accepted that under high power, continuous wave conditions radical chain growth takes place.¹⁵

WO08/082,293 is concerned with depositing an antimicrobial coating on a surface by plasma polymerisation of quaternary amines. Nanoparticle additives may be included in the polymer coating to provide UV protection, self-cleaning functionality or further improve antimicrobial functionality.

Hegemann et al¹⁶ suggest plasma polymerisation of acetylene mixed with ammonia in a process where both deposition and etching took place yielding a nanoporous, cross-linked network with accessible functional groups. They also suggest incorporating silver nanoparticles by sputtering a silver target.

US 2002/195950 describes the deposition copper (II) trifluoroacetylacetone on a polyester film in plasma, and alleges that the resulting film exhibits antibacterial properties. This document does not disclose plasma polymerisation of metal ion complexes having a polymerisable ligand.

There remains a need for improved antimicrobial coatings which can be formed on a range of substrates, and improved methods for their formation.

### SUMMARY OF THE INVENTION

Broadly, the present invention relates to materials and methods for forming a coating layer on a substrate by plasma deposition of metal ion complexes. The plasma deposition preferably includes plasma polymerisation of polymerisable ligands of the metal ion complexes.

The present inventors have found that these coatings can confer antimicrobial properties. For example, data presented herein demonstrates that coatings formed by plasma deposition of metal ion complexes are effective against Staphylococcus aureus, Pseudomonas aeruginosa, Micrococcus luteus, Saccharomyces cerevisiae and Escherichia coli, but are not cytotoxic to mouse or human cells.

It is believed that the antimicrobial properties of the coatings are conferred by the metal component of the metal ion complexes, which is incorporated into the coating. Accordingly, it will be understood that nature of the ligand(s) of the complexes is not particularly limited in the present invention. However, alternatively or additionally, the ligands themselves may provide antimicrobial properties.

Use of metal ion complexes is particularly beneficial because it provides a simple way of providing plasma deposited coatings with metals incorporated, avoiding techniques which can be difficult and time-consuming, such as handling nanoparticles or sputtering. Additionally, metal ion complexes often have vapour pressures which are ideally suited to plasma deposition, which takes place in the vapour phase.

Accordingly, in a first aspect the present invention provides a method of forming an antimicrobial coating layer on a substrate, comprising exposing a surface of the substrate and a plurality of metal ion complexes to a plasma environment, each metal ion complex comprising a metal ion having a ligand coordinated thereto, and thereby causing plasma deposition of an antimicrobial coating on the surface of the substrate.

It is believed that in some cases the ligands of the metal ion complexes can undergo polymerisation to form the coatings when exposed to a plasma environment. Accordingly, it may be preferable that the metal ion complexes comprise at least one polymerisable ligand, for example a plasma polymerisable ligand. Polymerisable ligands include those which have a moiety which can undergo polymerisation, such as plasma polymerisation. For example, the ligand may have a moiety which can undergo radical chain polymerisation when exposed to a plasma environment, for example an alkene or alkyne.

The plasma environment for causing plasma deposition of the antimicrobial coating may be provided by any apparatus suitable for generating plasma. Accordingly, in a further aspect the present invention provides use of a plasma generator for forming an antimicrobial coating on a surface of a substrate from a plurality of metal ion complexes.

The coating methods of the present invention are particularly useful as they can be quick and simple to perform. For example, the coatings could be applied in situ in hospitals, factories or other locations where it is desirable to provide antimicrobial coatings on a surface of a substrate. The methods of the invention may be used to replace or renew coatings on substrates which have been coated before, or to provide a first coating on a substrate which has not previously been coated. It will be understood that this coating or re-coating may form part of sterilization procedures taking place in the hospital, factory or other location. In a further aspect, then, the present invention provides use of a plasma generator for sterilizing a substrate.

The antimicrobial coatings formed by methods of the present invention are useful in conferring antimicrobial properties to a wide variety of substrates, including those made from woven fabric, non-woven fabric, plastic, glass and/or metal. It will be understood that the antimicrobial properties render the coatings particularly useful when formed on substrates which are used in medical or personal care applications, including hospital or other medical equipment, and products which are in contact with the body in, use. For example, the antimicrobial coatings may be employed to inhibit microbial colonization and/or infection. Substrates coated with the present coatings are particularly useful in preventing or treating skin disorders, for example those caused, exacerbated or irritated by microbes such as bacteria or fungi.

Accordingly, in a further aspect the present invention provides a substrate having an antimicrobial coating layer formed on a surface thereof, wherein the coating is formed by exposing a surface of the substrate and a plurality of metal ion complexes to a plasma environment, each metal ion complex comprising a metal ion having a ligand coordinated thereto, and thereby causing plasma deposition of an antimicrobial coating on the surface of the substrate. The present invention also provides an antimicrobial film, formed by a method of the present invention.

It will be understood that the coated substrates of the invention may be for use in a method of medical treatment, for example for the treatment and/or prophylaxis of microbial infection. The substrate may also be useful for the treatment and/or prophylaxis of skin disorders or disorders of mucous membranes. In a further aspect, then, the present invention provides use of a metal ion complex in the manufacture of a medicament for the treatment and/or prophylaxis of microbial infection. The present invention also provides use of a metal ion complex in the manufacture of a medicament for the treatment and/or prophylaxis of skin disorders or disorders of mucous membranes. It is understood that the medicament may be a coating or coated substrate of the present invention, for example a coated wound dressing, or a coated medical device such as an implantable medical device, for example a stent.

Additionally, it is believed that some of the metal ion complexes provided by the inventors for use in the present methods are new. Accordingly, in a further aspect the present invention provides a metal ion complex according to the general formula I,

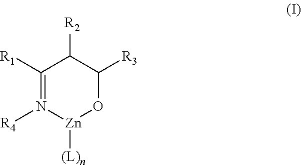

wherein
each R₁, R₂ and R₃ is independently H, substituted or unsubstituted, saturated or unsaturated C₁₋₁₀ alkyl, or R₁ and R₂ or R₂ and R₃ together form substituted or unsubstituted C₆₋₁₀ cycloalkyl, substituted or unsubstituted C₆₋₁₀ aryl or substituted or unsubstituted C₄₋₁₀ heteroaryl;
each R₄ independently is substituted or unsubstituted, saturated or unsaturated C₁₋₁₀ alkyl,
provided that at least one of R₁, R₂, R₃ and R₄ has a C—C double bond or a C—C triple bond;
and wherein n is an integer from 1-6, more preferably from 1 to 4 or from 1 to 2, and each L is a further ligand.

It will be understood that when R₁, R₂ or R₃ is substituted, the C—C double bond or C—C triple bond may be part of the substituent group.

The C₁₋₁₀ alkyl group of R₄ may optionally include one or more Si atoms in place of C atoms.

As discussed above, it is believed that the antimicrobial properties of the coatings are provided by the central zinc ion, and that polymerisability of the complexes is provided by the C—C double or C—C triple bond in one of R₁ to R₄. Accordingly, it will be understood that the nature of the further ligand(s) is not particularly limited in the present invention. However, it may be preferable that the further ligand(s) are selected so that the overall charge of the complex is zero. For example, it may be preferable that (L)ₙ includes one monoanionic ligand such as acetate and 0, 1 or 2 neutral ligands such as phosphine ligands.

In some embodiments, n may be 1 and L may be a second Schiff base ligand like the one illustrated in formula I above, and accordingly the present invention may provide metal ion complexes according to the general formula Ia,

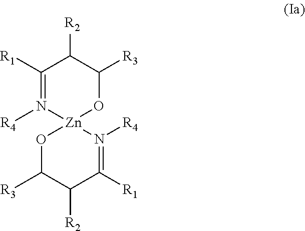

wherein
each R₁, R₂ and R₃ is independently H, substituted or unsubstituted, saturated or unsaturated C₁₋₁₀ alkyl, or R₁ and R₂ or R₂ and R₃ together form substituted or unsubstituted C₆₋₁₀ cycloalkyl, substituted or unsubstituted C₆₋₁₀ aryl or substituted or unsubstituted C₄₋₁₀ heteroaryl; and
each R₄ independently is substituted or unsubstituted C₁₋₁₀ alkyl having at least one C—C double bond or at least one C—C triple bond.

The C₁₋₁₀ alkyl of R₄ may optionally include one or more Si atoms in place of C atoms.

Alternatively, each further ligand (L) may be selected from monodentate, bidentate, tridentate or tetradentate ligands, more preferably from monodentate or bidentate ligands. Each further ligand may be selected from ligands wherein the donor atom(s) are selected from C, O, N, P, S and halogen atoms, or π-donor ligands such as alkenes, alkynes and aromatic species.

Example O ligands include H₂O; hydroxy; alkoxy ligands such as substituted or unsubstituted, saturated or unsaturated C₁₋₁₄ alkoxy; carboxylate ligands such as C₀₋₁₄ alkyl-C(O)O⁻; dicarboxylate ligands such as ⁻O(O)C—O₀₋₁₄ alkyl-C(O)O⁻; polycarboxylate ligands such as EDTA; dicarbonyl ligands such as acetylacetone; nitrate and nitrite.

Example N ligands include amines such as R₃N, wherein each R is independently H or substituted or unsubstituted, saturated or unsaturated C₁₋₁₄ alkyl, such as ammonia, trimethylamine and triethylamine; diamines such as C₁₋₁₄ alkyl diamine including ethylenediamine, triamines such as diethylene triamine and tetraamines such as triethylenetetraamine; and N-containing heteroaromatic ligands such as pyridine, pyrazine, pyrimidine, pyridazine, 2,2-bipyridine, isoquinoline, 1,8-naphthyridine, 1,10-phenanthroline and terpyridine; and cyano ligands such as C₁₋₁₄ alkyl-CN including Me-CN, and thiocyanate.

Example S ligands include sulphite; sulphide; and isothiocyanate.

Example C ligands include carbonyl (CO); and cyanide.

Example P ligands include phosphine ligands having the general formula PR₃, wherein each R is independently selected from substituted or unsubstituted, saturated or unsaturated C₁₋₁₀ alkyl, substituted or unsubstituted C₆₋₁₀ cycloalkyl, substituted or unsubstituted C₆₋₁₀ aryl and substituted or unsubstituted C₄₋₁₀ heteroaryl. Alternative P ligands include phosphite ligands having the formula P(OR)₃, wherein each R is independently selected from substituted or unsubstituted, saturated or unsaturated C₁₋₁₀ alkyl, substituted or unsubstituted C₆₋₁₀ cycloalkyl, substituted or unsubstituted C₆₋₁₀ aryl and substituted or unsubstituted C₄₋₁₀ heteroaryl.

Example halogen ligands include fluoride, chloride, bromide and iodide.

Example π-donor ligands include alkenes such as substituted or unsubstituted C₁₋₁₄ alkenes having 1 to 7, preferably 1 to 3 or 1 to 2 C—C double bonds; alkynes such as substituted or unsubstituted C₁₋₁₄ alkynes having 1 to 7, preferably 1 to 3 or 1 to 2 C—C triple bonds; and aromatic n-bond ligands including substituted or unsubstituted C₁₋₁₄ aromatic ring systems having 0 to 3 heteroatoms, such as benzene and cyclopentadiene.

It is understood that the complex illustrated by general formula I or Ia may exist in a dimeric form wherein one or more coordinating atoms is also coordinated to a second metal ion, for example the Zn central metal ion of a second complex according to formula I or Ia. For example, the complex illustrated by general formula Ia may exist in a dimeric form wherein one or both of the coordinating oxygen atoms is also coordinated to a second metal ion, for example the Zn central metal ion of a second complex according to general formula Ia, for example as illustrated in FIG. 2 (Ligand System 1).

In the metal ion complex illustrated by general formula I or Ia, the Zn central metal ion may be replaced with a Cu central metal ion, for example a Cu^{II} central metal ion.

The present invention also provides a metal ion complex according to the general formula II

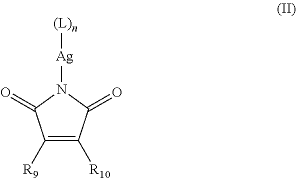

wherein
R₉ and R₁₀ are each independently selected from H and substituted or unsubstituted, saturated or unsaturated C₁₋₁₀ alkyl;
n is an integer from 1-6, more preferably from 1 to 4 or from 1 to 3 from 1 to 2; and
each L is a further ligand, for example as defined above.

As above, it will be understood that the nature of the further ligand(s) is not particularly limited. However, it is preferable that the ligands are selected so that the overall charge of the complex is zero. For example, it may be preferable that (L)ₙ includes 1, 2, 3 or 4 neutral ligands.

Neutral ligands are understood to include species which form dative bonds with the central metal ion, including those having one or more atoms which can each donate two electrons to the metal ion, and those which can donate n electrons to the central metal ion, such as alkenes, alkynes and aromatic ligands.

The neural ligands may contain one or more donor atoms which are capable of donating electron density to the central metal ion. Example donor atoms include C atoms, N atoms, O atoms, S atoms and P atoms. Accordingly, preferred neutral ligands include C ligands, N ligands, O ligands, S ligands, P ligands and n-donor ligands.

Example neutral O ligands include H₂O.

Example neutral N ligands include amines such as R₃N, wherein each R is independently H or substituted or unsubstituted, saturated or unsaturated C₁₋₁₄ alkyl, such as ammonia, trimethylamine and triethylamine; diamines such as C₁₋₁₄ alkyl diamine including ethylenediamine, triamines such as diethylene triamine and tetraamines such as triethylenetetraamine; and N-containing heteroaromatic ligands such as pyridine, pyrazine, pyrimidine, pyridazine, 2,2-bipyridine, isoquinoline, 1,8-naphthyridine, 1,10-phenanthroline and terpyridine; and cyano ligands such as C₁₋₁₄ alkyl-CN including Me-CN.

Example neural C ligands include carbonyl (CO).

Example neutral P ligands include phosphine ligands having the general formula PR₃, wherein each R is independently selected from substituted or unsubstituted, saturated or unsaturated C₁₋₁₀ alkyl, substituted or unsubstituted C₆₋₁₀ cycloalkyl, substituted or unsubstituted C₆₋₁₀ aryl and substituted or unsubstituted C₄₋₁₀ heteroaryl.

Alternative P ligands include phosphite ligands having the formula P(OR)₃, wherein each R is independently selected from substituted or unsubstituted, saturated or unsaturated C₁₋₁₀ alkyl, substituted or unsubstituted C₆₋₁₀ cycloalkyl, substituted or unsubstituted C₈₋₁₀ aryl and substituted or unsubstituted C₄₋₁₀ heteroaryl. It may be preferred that each L is a phosphite ligand of the formula P(OR)₃. Where at least one L is a phosphite ligand, it maybe preferred that n=3. It is particularly preferred that n=3 and each L is a phosphite ligand.

Particularly preferred phosphite ligands are those where each R is alkyl substituted or unsubstituted, saturated or unsaturated C₁₋₁₀ alkyl, more preferably unsubstituted, saturates alkyl. C₁₋₅ alkyl is particularly preferred, such as ethyl.

Example neutral n-donor ligands include alkenes such as substituted or unsubstituted C₁₋₁₄ alkenes having 1 to 7, preferably 1 to 3 or 1 to 2 C—C double bonds; alkynes such as substituted or unsubstituted C₁₋₁₄ alkynes having 1 to 7, preferably 1 to 3 or 1 to 2 C—C triple bonds; and aromatic π-bond ligands including substituted or unsubstituted C₁₋₁₄ aromatic ring systems having 0 to 3 heteroatoms, such as benzene and cyclopentadiene.

The metal ion complex may have the general formula IIa

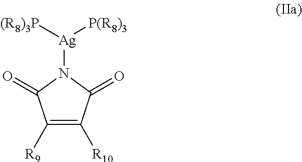

wherein
each R₈ is independently selected from substituted or unsubstituted, saturated or unsaturated C₁₋₁₀ alkyl, substituted or unsubstituted C₆₋₁₀ cycloalkyl, substituted or unsubstituted C₆₋₁₀ aryl and substituted or unsubstituted C₄₋₁₀ heteroaryl; and
R₉ and R₁₀ are each independently selected from H and substituted or unsubstituted, saturated or unsaturated C₁₋₁₀ alkyl.

Unless otherwise stated, the term “substituted” as used herein includes substitution with OH, carbonyl, C₁₋₁₀ alkyl (preferably C₁₋₄ alkyl) and halogen, (preferably F). Alkyl substituents may further be substituted with up to three, four or five substituents selected from OH and F.

Where an Si atom replaces a C atom in any of the formulae disclosed herein, it will be understood that the Si atom may form part of an Si(R₁₅)₂ group, where R₁₅ is as defined herein.

Embodiments of the present invention will now be described in more detail by way of example and not limitation with reference to the accompanying figures.

### BRIEF DESCRIPTION OF THE FIGURES

FIG. 1 shows example metal ion complexes useful in the present invention.

FIG. 2 shows some metal ion complexes useful in the present invention and example syntheses, wherein M²⁺ can represent Cu²⁺ or Zn²⁺.

FIG. 3 shows (A) FT-IR spectrum for the metal ion complex PSSM and (B) FT-IR spectrum for a coating formed by plasma deposition of PSSM.

FIG. 4 shows a SEM image of a PSSM coating on a thin gold film.

FIG. 5 shows the zone of inhibition around pellets of PSSM monomer for S. aureus (A), M. luteus (B) and P. aeruginosa (C), after 24 h at 37° C.

FIG. 6A shows colonisation by M. luteus after 24 hours growth in LB media on a PSSM coated Petri dish (left) and an untreated Petri dish (right). 6B shows the results of an assay for measurement of bacterial cell viability.

FIG. 7 shows the results of a study of S. aureus growth on Petri dishes coated with a plasma deposited PSSM coating, using a BAC-light Live-Dead stain.

FIG. 8 shows the effect of PSSM plasma deposited coatings on S. cerevisiae.

FIG. 9 shows the effect of PSSM plasma coated non-woven squares on E. coli growth.

FIG. 10 shows end point measurements after 24 hours for both S. aureus and P. aeruginosa on untreated, PSSM coated and ZSB coated Petri dishes.

FIG. 11 shows the effect of ZSB plasma coated polypropylene non-woven on S. aureus.

FIG. 12 shows the effect of ZSB coated non-woven on S. aureus and P. aeruginosa.

FIG. 13 shows time course measurements of bacterial growth on ZSB plasma coated Petri dishes.

FIG. 14 shows the effect of ZSB monomer powder on S. aureus and M. luteus.

FIG. 15 shows the results of Cytotoxicity tests on ZSB and PSSM plasma coated coverslips on Swiss mouse fibroblasts.

FIG. 16 shows the results of Cytotoxicity tests on ZSB and PSSM plasma coated coverslips on human neonatal epidermal keratinocytes.

FIG. 17 shows the effect of the plasma deposition process on non-wovens.

FIG. 18 shows inhibitory concentration curves for (A) ZSB and (B) CSB against S. aureus and P. aeruginosa.

FIG. 19 shows time course measurements for bactrieal growth on fabrics coated with plasma polymerised ZSB and noncoated fabrics, (A) against P. aeruginosa and (B) against S. aureus.

FIG. 20 shows (A) a SIMS map of ⁶⁴Zn²⁺ in plasma polymerised ZSB film and (B) a SIMS map of organic fragment C₃H₁₆N⁺.

FIG. 21 shows transition electron microscopy images of (A) plasma polymerised ZSB and (B) plasma polymerised CSB, deposited using a pulse plasma.

FIG. 22 shows the live:dead ratio for (A) P. aeruginosa and (B) S. aureus on plasma polymerised CSB films, under continuous wave and different pulsed plasma conditions.

FIG. 23 shows (A) FTIR spectrum of PTSM and (B) FTIR spectrum of PTSM plasma polymerised at 50 W on 1/40 ms cycle for 30 minutes.

FIG. 24 shows a SIMS map and spectra of PTSM plasma polymerised at 50 W on 1/40 ms cycle for 30 minutes.

FIG. 25 shows the action of plasma polymerised PTSM film with a fluorinated adlayer against P. aeruginosa.

FIG. 26 shows the concentration of P. aeruginosa eluted through uncoated filter media and filter media coated with ZSB.

### DETAILED DESCRIPTION

### Metal Ion Complexes

As discussed above, in the present invention employs metal ion complexes in the formation of antimicrobial coatings. The term “metal ion complexes” as used herein includes compounds having a single central metal ion with one or more ligands coordinated thereto. The ligands may be any chemical species capable of coordinating to the metal ion, for example by acting as an electron donor (Lewis base) to the metal ion.

Preferably, the metal ions in the metal ion complex are antimicrobial metal ions. The term “antimicrobial metal ion” in this context includes metal ions which are antimicrobial when in their ionic state, and also ions of metals which are antimicrobial when in their metallic state. The term “antimicrobial” as used herein is understood to apply to substances including those which inhibit microbial attachment to surfaces, kill microbes and/or inhibit microbial reproduction. The term microbe is understood to include all microorganisms, such as bacteria, fungi, archaea and protists, in particular bacteria and fungi such as yeast. The terms “microbial” and “antimicrobial” should be interpreted accordingly. Example microbes which the coatings of the present invention may be effective against include Staphylococcus sp., such as S. aureus, Pseudomonas sp., such as P. aeruginosa, Micrococcus sp., such as M. luteus, Saccharomyces sp., such as S. cerevisiae, Candida sp., such as C. albicans, Staphylococcus sp., such as S. epidermis, Streptococcus sp., such as S. pyrogenes, Klebsiella sp. and Escherichia sp., such as E. coli.

Suitable metal ions for the metal ion complexes include ions of Ag, Zn, Cu, Au, Pt and Bi. The ions may preferably be selected from Ag⁺, Zn²⁺, Cu⁺, Ce²⁺, Au⁺, Au³⁺, Pt²⁺, Pt⁴⁺ and Bi³⁺. As discussed above, the use of metal ion complexes is particularly beneficial because metal ion complexes often have vapour pressures which are ideally suited to plasma deposition. As discussed in more detail below, the plasma deposition process may be done in the vapour phase. Accordingly, it may be preferable that the metal ion complex has a suitable volatility for it to vaporise under the operating conditions of the plasma process. The volatility of the metal ion complex is not particularly limited, because conditions such as temperature and pressure can be modified and optimised for a given monomer to enable the process to be carried out in the vapour phase. However, it may be preferred, for example, that the metal ion complexes readily vaporise at 0.1 Pa, at 25° C.

In particular, it may be preferable that the metal ion complexes have an equilibrium vapour pressure greater than or equal to 0.1, 1, 5, 10 or 50 Pa, and/or an equilibrium vapour pressure less than or equal to 20, 15, 10, 8, 6, 4, 2 or 1 KPa, at 25° C.

It may be preferable that the metal ion complexes have a significant vapour pressure at 0.4 mTorr. For example, a significant vapour pressure may mean that the compounds show significant mass loss in a thermogravimetric analyser at a temperature between 50° C. and 250° C. For example, the mass loss may be at least 10%, at least 20%, at least 30%, at least 40% or at least 50% mass loss.

Melting point is related to equilibrium vapour pressure, with lower melting point metal ion complexes in general having a higher equilibrium vapour pressure. The present inventors have found that it may be preferable that the metal ion complexes have a melting point less than or equal to 200, 150, 140, 130 or 120° C.

The vapour pressure of the metal ion complex may be modified by modifying the chemical structure of the ligand(s) of the metal ion complex. For example, the ligand(s) may be fluorinated, which can increase their volatility.

As discussed above, the nature of the ligand(s) of the metal ion complexes is not particularly limited in the present invention. It is preferred that each metal ion complex comprises at least one ligand which is polymerisable. Preferably, the ligand is plasma polymerisable.

Each polymerisable ligand may include a polymerisable moiety, such as a moiety which can undergo radical chain polymerisation when exposed to a plasma environment. Accordingly, it is may be preferable that the polymerisable ligand includes a C—C double bond or a C—C triple bond.

It may be preferable that the polymerisable moiety is polymerisable when the ligand is coordinated to the metal ion. For example, it may be preferable that the polymerisable moiety, such as a C—C double bond or a C—C triple bond, does not form part of the coordination moiety.

Preferably, the polymerisable ligand includes a coordination moiety suitable for coordinating the ligand to the metal ion. Preferably the coordination moiety is selected from acyl, imide, amine, carbonyl, dicarbonyl, cyanyl, nitro, a Schiff-base and hydroxyl, preferably from acyl and a Schiff-base.

As used herein, the term “Schiff-base” includes moieties having the general structure —N═CRₐR_{b}, wherein Rₐ and R_{b} are each independently selected from H, optionally substituted, saturated or unsaturated C₁₋₁₀ alkyl, and optionally substituted C₆₋₁₀ aryl. The C₁₋₁₀ alkyl and C₆₋₁₀ aryl may each be substituted with one or more, preferably up to three, four or five substituents independently selected from F, OH, carbonyl, C₁₋₄ alkyl having up to three F substituents and C₆₋₁₀ aryl having up to five F substituents.

Some preferred Schiff-base moieties include those wherein Rₐ is aryl optionally substituted with up to three substituents selected from —OH and F, and/or R_{b} is H, Me or Et, optionally substituted with up to three F atoms.

The polymerisable ligand may be maleimide optionally substituted with up to two substituents. Preferably, the maleimide ligand is not substituted, or is substituted with up to two substituents selected from F, methyl and trifluoromethyl.

The polymerisable ligand may alternatively be a ligand having the general formula III

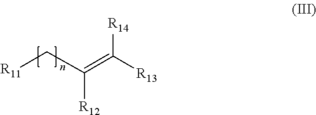

wherein
n is an integer from 0 to 14, preferably from 0 to 10, more preferably from 1 to 6, or may be from 0 to 5, for example from 0 to 2 or 0 to 1;
R₁₁ is a coordination moiety suitable for coordinating the ligand to the metal ion, which preferably includes a moiety selected from acyl, imide, amine, carbonyl, dicarbonyl, cyanyl, nitro, a Schiff-base and hydroxyl, preferably from acyl, dicarbonyl and a Schiff-base; and
each of R₁₂, R₁₃ and R₁₄ is independently selected from H, F, C₁₋₁₀ alkyl, C₆₋₁₀ aryl and C₅₋₁₀ heteroaryl, preferably from H, F, C₁₋₄ alkyl and phenyl, more preferably from H, methyl and phenyl, wherein each alkyl and aryl is optionally substituted, for example with up to five substituents. Preferred substituents include OH and F. For example, each of R₁₂, R₁₃ and R₁₄ may optionally have one OH substituent selected, and up to three, up to two or up to one F substituents.

In formula III, one or more of the CH₂ groups within [ ]ₙ may optionally be replaced by Si(R₁₅)₂, wherein each R₁₅ is independently H or saturated or unsaturated, substituted or unsubstituted C₁₋₁₀ alkyl.

Silane containing ligands (e.g. wherein one or more of the CH₂ groups within [ ]ₙ may be replaced by Si(R₁₅)₂) may be preferred as they may impart antifouling properties to the coatings.

Particularly preferred R₁₁ coordination moieties include:

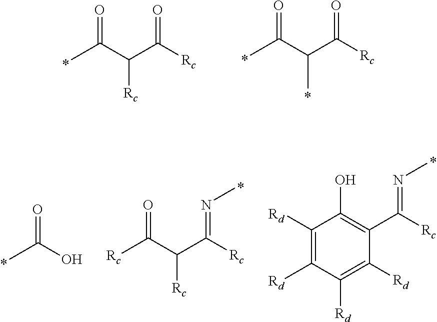

wherein the asterisk denotes the point of attachment to the rest of the ligand; R_{c} is selected from H, C₁₋₄ alkyl and C₆₋₁₀ aryl, wherein the alkyl and aryl are optionally substituted with F; and R_{d} is H, C₁₋₆ alkyl and C₆₋₁₀ aryl.

Example polymerisable ligands include maleimide, acrylate tiglate, crotonate, cinnamate and the Schiff-base ligands having the structures:

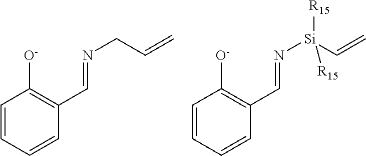

and fluorinated analogues thereof, wherein R¹⁵ is as defined above. The fluorinated analogues may have up to five, up to four, up to three, up to two, or one F or CF₃ substituents on the ligand.

The metal ion complexes may have one, two or more polymerisable ligands. Where the metal ion complexes have more than one polymerisable ligand, each polymerisable ligand is selected independently. The metal ion complexes may include one, two or more further ligands in addition to the polymerisable ligand(s). Again, it will be understood that the nature of the further ligands is not particularly limited, and any ligand may be used. Example ligands include bipyridine and phosphine ligands. The phosphine ligands may have the general formula PR₃, wherein each R group is independently selected from C₁₋₁₀ alkyl, aryl and heteroaryl, preferably from methyl, ethyl, propyl, butyl, cyclohexanyl and phenyl. Other example ligands include those listed above with reference to formulas I, Ia, II and IIa.

The metal ion complexes may have a structure according to general formula I as defined above, or according to general formula Ia:

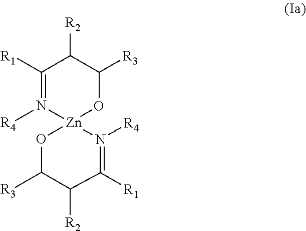

wherein
each R₁, R₂ and R₃ is independently H, substituted or unsubstituted, saturated or unsaturated alkyl, or R₁ and R₂ or R₂ and R₃ together form substituted or unsubstituted C₆₋₁₀ cycloalkyl, substituted or unsubstituted C₆₋₁₀ aryl or substituted or unsubstituted C₄₋₁₀ heteroaryl; and
each R₄ is independently substituted or unsubstituted alkyl having at least one C—C double bond or at least one C—C triple bond.

In formula Ia, the C₁₋₁₀ alkyl of R₄ may optionally include one or more Si atoms in place of C atoms.

In formula I or Ia, for example R₁ may be H or C₁₋₄ alkyl optionally substituted with up to three substituents, for example selected from OH and F. R₂ and R₃ may together form phenyl optionally substituted with F, OH or C₁₋₄ alkyl optionally substituted with up to three substituents selected from OH and F.

In formula I or Ia, R₄ may have the structure:

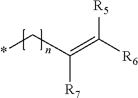

wherein
n is an integer from 0 to 10, preferably from 0 to 5, more preferably from 0 to 2 or 0 to 1;
and
each of R₅, R₆ and R₇ is independently selected from H, F, C₁₋₁₀ alkyl, preferably from H, F, C₁₋₄ alkyl, more preferably from H, methyl and ethyl, wherein each alkyl is optionally substituted, for example with up to three substituents. Preferred substituents include OH and F.

In the above formula, one or more of the CH₂ groups within [ ]ₙ may optionally be replaced by Si(R₁₅)₂, wherein each R₁₅ is independently H or saturated or unsaturated, substituted or unsubstituted C₁₋₁₀ alkyl.

As noted above, in formula I or Ia, the Zn ion may be replaced with a Cu ion, preferably Cu^{II}.

Example metal ion complexes according to general formula I include the compound having the structure:

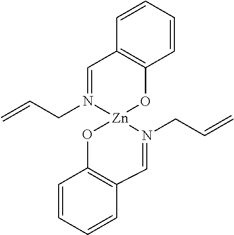

and fluorinated analogues thereof. The fluorinated analogues may have up to five, up to four, up to three, up to two, or one F or CF₃ substituents on each ligand. The metal ion complex shown above is referred to as ZSB herein.

It will be understood that a silane analogue of this metal ion complex having the silane-containing Schiff base ligand shown above is also useful in the present invention.

An alternative metal ion complex is the compound having the structure:

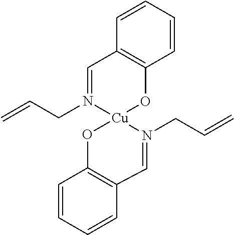

and fluorinated analogues thereof. The fluorinated analogues may have up to five, up to four, up to three, up to two, or one F or CF₃ substituents on each ligand. The metal ion complex shown above is referred to as CSB herein.

It will be understood that a silane analogue of this metal ion complex having the silane-containing Schiff base ligand shown above is also useful in the present invention.

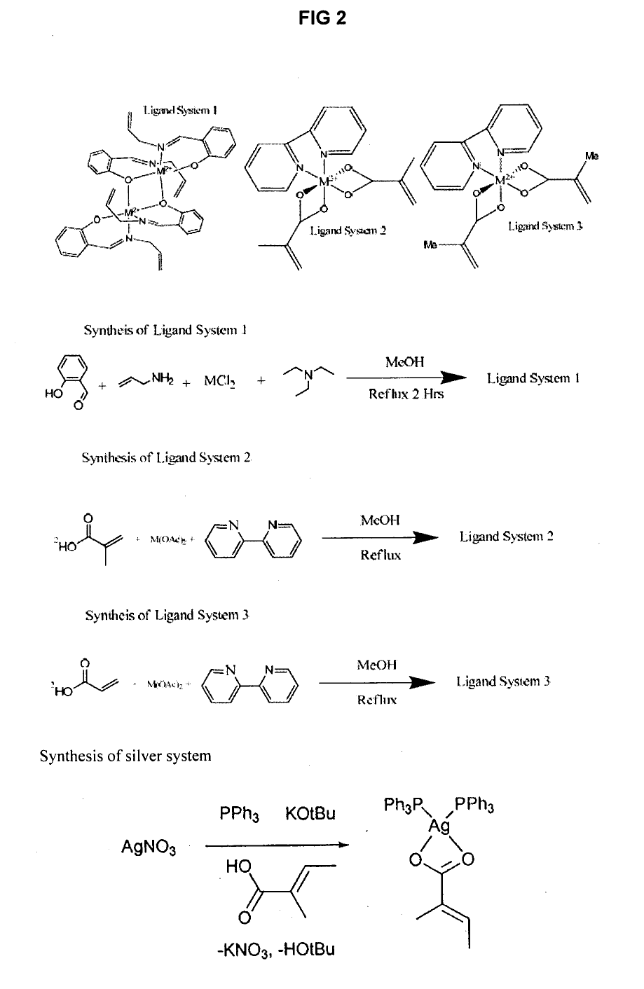

It is understood that the ZSB complex may exist in a dimeric form wherein one or both of the coordinating oxygen atoms is also coordinated to a second metal ion, for example the Zn central metal ion of a second ZSB complex, as illustrated in FIG. 2 (Ligand System 1).

The metal ion complexes may have a structure according to general formula II as defined above, or according to general formula IIa

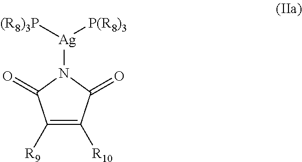

wherein
each R₈ is independently selected from substituted or unsubstituted, saturated or unsaturated C₁₋₁₀ alkyl, C₆₋₁₀ cycloalkyl, C₆₋₁₀ aryl and C₄₋₁₀ heteroaryl; and
R₉ and R₁₀ are each independently selected from H and substituted or unsubstituted, saturated or unsaturated C₁₋₁₀ alkyl.

Preferably, R₈ is selected from methyl, ethyl, propyl, butyl, cyclohexyl and phenyl. Preferably, R₉ and R₁₀ are selected from H, methyl and trifluoromethyl.

An example metal ion complex according to general formula II has the structure:

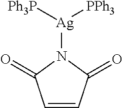

This complex is referred to as PSSM herein.

Another example metal ion complex according to general formula II has the structure

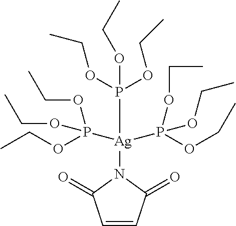

This complex is referred to as PTSM, or as tris-triethylphosphito silver maleimide, herein.

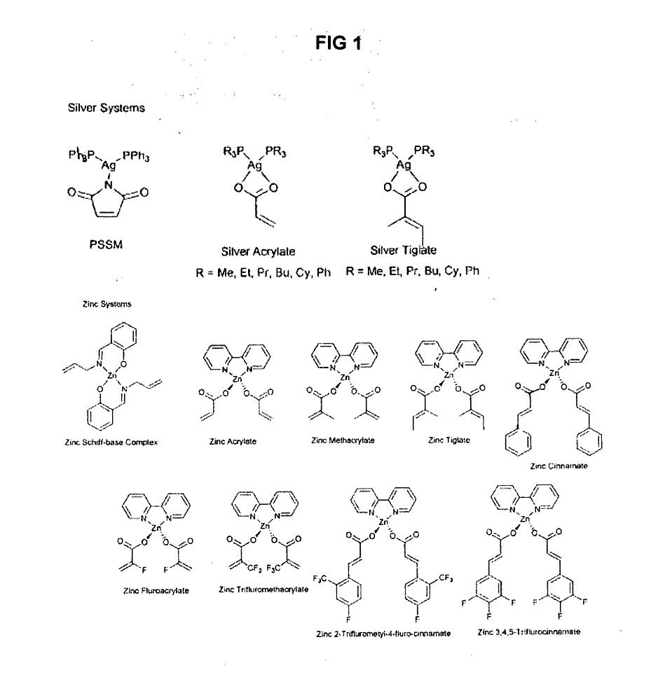

Other example metal ion complexes according to the present invention are shown in FIG. 1.

### Plasma Deposition

The plasma deposition methods of the present invention comprise exposing the plurality of metal ion complexes to a plasma environment. The plasma deposition methods may be conducted in the vapour phase.

The term “plasma environment” as used herein includes an environment wherein a plasma is generated. A plasma is understood to include a vapour phase mixture which may contain radicals, ions, electrons and photons. Accordingly, a plasma environment includes an environment wherein the metal ion complexes become ionised or radicalised.

The step of exposing the metal ion complexes to a plasma environment may comprise applying power to the metal ion complexes, for example electromagnetic power such as radio frequency power. Preferably, the power supplied is greater than or equal to 1, 2, 5, 10, 20 or 50 W, and less than or equal to 200, 150 or 100 W. The frequency of the radio frequency power may be, for example, in the range from 3 Hz to 300 GHz. In some cases, it is preferred that the power supplies is less than or equal to 90, 80, 70, 60, 50, 40, 30, or 10 W. With power supply less than or equal to these values, the structure of the ligands may be better conserved in the coating than when plasma deposition is carried out at higher power. This may lead to improved antimicrobial properties.

The power may be supplied by electrodes. The input impedance of the power may be adjusted, for example using a matching unit, to reduce reflected power. As discussed in more detail below, during the plasma deposition process, the metal ion complexes may be contained within or passing through a chamber. The electrodes for providing the power may be located externally of the chamber.

It may be preferable that the power supplied to the metal ion complexes continuously, for example as a continuous wave. Alternatively, the power supplied to the metal ion complexes may be pulsed.

When pulsed power supply is used, the structure of the ligands may be better conserved in the coating than when plasma deposition is carried out in a continuous. This my lead to improved antimicrobial properties. It may be preferred that the pulses of power are less than or equal to the length of the pauses between the pulses of power supply. For example, the pulse to pause ratio could be less than or equal to 1:1, 1:2, 1:3, 1:5, 1:10, 1:15, 1:20, 1:25, 1:30, 1:35, 1:40, 1:45 or 1:50. Preferred pulse to pause ratios are about 1:40, for example 1:30 to 1:50.

It may be preferred that the pause between pulses is from 10 to 100 milliseconds, for example 30 to 50 milliseconds, for example about 40 milliseconds. For example, the pulsing sequence may comprise a 1 millisecond power pulse followed by a 40 millisecond pause, followed by a 1 millisecond power pulse followed by a 40 millisecond pause, and so on.

It may be preferable that during the plasma deposition process, the metal ion complexes flow over the substrate to be coated. For example, the substrate may be held within a chamber, and the metal ion complexes caused to flow through the chamber. The present inventors have found that flow rates of between 1 and 10 cm³ min⁻¹ at 20° C. are particularly suitable for the plasma deposition process. Preferably, the flow rate is greater than or equal to 0.1, 0.5, 1 or 2 cm³ min⁻¹, and less than or equal to 50, 40, 30, 20 or 10 cm³ Particularly preferred are flow rates between 1 and 10 cm³ min⁻¹.

The metal ion complexes may be caused to flow into the chamber by reducing the pressure in the chamber, for example by connecting it to a vacuum source. Flow of the metal ion complexes into the chamber may be controlled by a valve. The source of metal ion complexes for flowing into the chamber may be, for example, solid metal ion complexes or liquid metal ion complexes, which can vaporise to form a vapour of metal ion complexes.

The pressure in the chamber is not particularly limited. Preferably, the pressure is low enough to enable the metal ion complexes to vaporise and pass into the chamber. Particularly preferred pressures are greater than or equal to 0.01, 0.05 or 0.1 Pa, and/or less than or equal to 1.0. 0.8, 0.6, 0.5, 0.4, 0.3 or 0.2 Pa.

Preferably, the chamber is substantially free of gases other than the vaporised metal ion complexes. This can be achieved for example by first cleaning the chamber by exposing it to an oxygen plasma and/or by evacuating the chamber prior to allowing the metal ion complexes to flow into the chamber. The term substantially free as used herein means that in addition to the metal ion complexes, the chamber contains only unavoidable impurities, resulting for example from residual air which remains in the chamber.

As discussed above, the present invention also provides use of a plasma generator for forming an antimicrobial coating on a surface of a substrate from a plurality of metal ion complexes. Preferably, the generator is capable of carrying out plasma deposition as described above. Accordingly, it will be understood that the plasma generator may comprise a chamber for holding the metal ion complexes and electrodes for supplying power to the metal ion complexes to create the plasma environment. The plasma generator may further comprise an input channel to allow the metal ion complexes to enter the chamber, which may have a valve. It may comprise a vacuum source to reduce the pressure in the chamber and/or an impedance matching unit to adjust the impedance of the power to reduce reflected power.

Without wishing to be bound by theory, it is believed that exposing the metal ion complexes to the plasma environment causes plasma polymerisable moieties of the polymerisable ligands, such as alkene or alkyne moieties, to become radicalised. It is believed that this causes the metal ion complexes to undergo radical chain polymerisation, which is analogous to the radical chain polymerisation of ethene. It is believed that surface ablation of the substrate also occurs.

It is believed that the plasma deposition process may also have the effect of sterilising the surface of the substrate which is exposed to the plasma environment. This could be due, in part, to the surface ablation which is believed to take place. Accordingly, deposition may for example be employed as a final stage in a manufacturing process of a coated substrate, or may be employed in situ for example in a hospital, factory or other location as described above, for example to replace or supplement routine sterilisation processes.

The plasma deposition methods described herein may further include, after plasma deposition of an antimicrobial coating on the surface of the substrate, exposing the treated surface to a plasma of fluorine-substituted hydrocarbon. For example, the plasma may be a plasma of fluorine substituted C₁₋₁₀ alkanes, alkenes or alkynes, preferably alkanes. More preferred are fluorine substituted C₁₋₄ alkanes. For example, each hydrocarbon may have less than or equal to 20 fluorine substituents, for example less than or equal to 15 or 10 fluorine substituents.

Most preferred is hexafluoroethane, C₂F₆.

Preferably the exposure to the plasma of fluorine-substituted hydrocarbon lasts for less than or equal to 5 minutes, more preferable less than or equal to 4 minutes, 3 minutes, 2 minutes, 1 minute, 50 seconds, 40 seconds or 30 seconds.

### Antimicrobial Coatings

The present invention provides antimicrobial coatings which can be formed on a variety of substrates. Possible substrates which could be coated are discussed below, under the heading “Applications of Antimicrobial Coatings”.

The nature of the coatings formed by the methods of the present invention is not fully understood. As demonstrated below in the examples, SEM images appear to show that nanoparticles derived from the metal ion complexes form in the coatings. Additionally, infrared (IR) spectra of the deposited coatings appear to show retention of ligand-metal ion bonds, suggesting that the metal ion complexes used to form the coating are not entirely dissociated in the plasma deposition process.

The films formed may have a thickness from about 5 nm to about 200 nm, more preferably from about 5 nm to about 150 nm, and most preferably from about 10 nm to about 50 nm or to about 100 nm. The thickness of the coatings can be determined by Surface Plasmon Resonance (SPR), for example as described below in the examples.

### Applications of Antimicrobial Coatings

The examples below demonstrate that the coatings can be formed on glass, gold-coated glass, polystyrene Petri dishes and polypropylene non-woven fabric. Additionally, the examples demonstrate that the coating process does not cause a structural change in the polypropylene non-woven fabric. Accordingly, it will be understood that the substrates which can be coated according to the present invention are not particularly limited. Coatings on substrates which comprise woven fabric, non-woven fabric, plastic, glass and/or metal may be preferable. Particularly preferred are substrates having a non-woven fabric component.

The antimicrobial nature of the coatings makes them particularly suitable to be applied to substrates for use in medical or personal care applications. In particular, the coatings are particularly useful on substrates which are in contact with the body, for example with skin or mucous membrane, in normal use.

For example, microbial growth is a particular problem when skin or mucous membrane is covered, for example by a wound dressing, nappy or underwear. As soon as skin or mucous membrane becomes covered, the environmental conditions for microbial growth improve. Microbes present on the covered skin or mucous membrane can multiply at enhanced rates, particularly when the environment is moist and/or not exposed to air. Secretions from these microbes include acid or alkali excretions which can alter the pH of the skin, toxin secretion and enzyme secretion, including protease secretion. These secretions and excretions can cause skin and mucous membrane irritation, and in the more severe cases skin or mucous membrane breakdown, such as dermatitis.

Particular conditions which can occur following to the covering of skin or mucous membrane include thrush. Thrush is a fungal infection, by the Candida genus of yeast, particularly Candida albicans. Symptoms include itching, burning and soreness, and inflammation of the infected area. The wearing of sanitary towels, incontinence pads, nappies and/or tight underwear can produce conditions favourable to Candida growth, which can lead to thrush. The coatings of the present invention have been found to be effective against fungi such as yeast, and accordingly it will be understood that providing the coatings of the invention on the above mentioned items may enable the treatment and/or prophylaxis of thrush.

Similarly, contact dermatitis (commonly known as nappy rash) may be caused by the wearing of incontinence pads or nappies. Damp or wet skin loses its structure, high pH can promote bacterial growth and the bacteria can secrete enzymes which break down the skin tissue. This environment can also promote or exacerbate pressure ulcers (commonly known as bed sores), which are particularly problematic when they become infected. The coatings of the present invention have been found to be effective against bacteria, and accordingly it will be understood that providing the coatings of the invention on tampons, sanitary towels, incontinence pads or nappies may enable the treatment and/or prophylaxis of contact dermatitis and/or pressure ulcers.

For similar reasons, contact dermatitis and yeast infections can occur under medical dressings, for example dressings for wounds and burns. An additional consideration with medical dressings is the need to prevent bacterial infection of the wound or burn. When skin is burnt, a large amount of tissue may be damaged which can reduce or destroy the natural barrier properties of skin, and wounds which break the skin also affect the barrier properties of skin. This can lead to opportunistic infection that can delay healing, and to septic shock. Additionally, microbial infection, particularly bacterial infection, can be a problem after surgery. The use of medical or surgical devices, for example implantable medical devices, which are coated with the present antimicrobial coatings may help to prevent or treat post-surgical infection. Accordingly, it will be understood that providing the coatings of the invention on dressings for wounds and/or burns may enable the treatment and/or prophylaxis of contact dermatitis and/or microbial infection.

The metal ion complexes, then, can be used in the manufacture of a medicament for the treatment and/or prophylaxis of microbial infection, and/or of skin or mucous membrane disorders such as inflammation and dermatitis. In particular, the antimicrobial coatings may be useful for the treatment and/or prophylaxis of infection of a wound, infection of a burn, infection of a pressure ulcer, post-surgical infection, thrush, contact dermatitis and pressure ulcers. The microbial infection may be by any microbe, in particular bacteria and/or yeast such as Staphylococcus sp., such as S. aureus, Pseudomonas sp., such as P. aeruginosa, Micrococcus sp., such as M. luteus, Saccharomyces sp., such as S. cerevisiae, Candida sp., such as C. albicans, Staphylococcus sp., such as S. epidermis, Streptococcus sp., such as S. pyrogenes, Klebsiella sp. and Escherichia sp., such as E. coli.

The medicament may be a substrate coated by the coating methods of the present invention. For example, then, the medicament may be a coated substrate such as a coated medical device, for example an, implantable medical device. Examples include a surgical seed, catheter (such as a urinary catheter, a vascular access catheter, an epidural catheter), a vascular access port, an intravascular sensor, a tracheotomy tube, a percutaneous endoscopic gastrostomy tube, an endotracheal tube, an implantable prosthetic device, such as a stent and related short-indwelling or biocontacting devices. The medicament may be a coated substrate such as a coated nappy, sanitary towel, tampon, incontinence pad, dressing such as a wound or burn dressing, bandages or underwear. Many of these substrates (particularly nappies, sanitary towels, incontinence pads and dressings such as wound or burn dressings) comprise a non-woven fabric component, which may be in contact with skin or mucous membrane in normal use. The present inventors have demonstrated that the coatings and coating methods of the present invention are particularly suited to non-woven fabric substrates.

As used herein, the term “non-woven fabric” includes fabrics or textiles formed from a web of fibres. In non-woven fabric, the fibres are not woven or knitted. Nonwovens are typically manufactured by putting small fibers together in the form of a sheet or web, and then binding them mechanically. Example non-woven fabrics include polypropylene non-wovens.

It will be understood that the manufacturing process of the medicament may include providing an antimicrobial coating on a substrate. Accordingly, the manufacture of the medicament may comprise any of the steps of the methods described herein for providing antimicrobial coatings.

The present invention also provides substrates coated by the present methods. The coated substrates may be for use in a method of medical treatment, and include the coated substrates mentioned above as possible medicaments. It will be understood that the present invention also provides a method of medical treatment for the treatment and/or prophylaxis of microbial infection and/or of disorders of the skin or mucous membrane, and the use of the present coated substrates in such methods. The coating methods of the present invention are applicable to coating the substrates mentioned herein, as medicaments or otherwise.

As well as the applications described above, the antimicrobial coatings may also be provided on other equipment for use in medical applications, for example in hospitals. There is significant interest in controlling infection in hospitals, in particular bacterial infection such as MRSA and Clostridium difficile. As discussed above, microbial colonisation of surfaces is a particular problem. However, the present coatings have been found to be effective against many species of microbe, and so it will be understood that providing the present antimicrobial coatings on the surface of hospital equipment may be beneficial. Accordingly, substrates which may be coated according to the present invention include medical equipment and devices which contact the body or body fluids in normal use. For example, suitable substrates include tubes, fluid bags, catheters, syringes and surgical equipment such as scalpels and forceps etc. Additionally, other equipment, for example equipment used in hospitals (e.g. healthcare equipment) may be coated according to the present invention, for example gowns (e.g. surgical gowns), surgical masks, protective gloves (e.g. surgical and examination gloves), curtains, uniforms and bedding such as pillow cases, waterproof mattress covers (for example in babies cots and intensive care beds) and sheets.

Alternative healthcare equipment includes surgical draperies, surgical socks, furniture such as tables including bedside tables, beds, and seating surfaces, and other equipment including storage containers, filters, and service trays. Additionally, the coatings of the invention are useful in coating equipment which it is desirable to keep free of microbes, for example equipment which is used in processing of food, for example kitchen equipment and surfaces, and factory equipment used in the manufacture or processing of food. For example, substrates which can be coated according to the present invention include containers (such as food storage containers), conveyors, blades, mixers, rollers and kitchen utensils (such as cutting and serving implements). Additional substrates include food preparation surfaces, flexible and rigid packaging and door handles.

Additionally, protective clothing worn by workers, for example overalls, gloves, masks and hats could be coated. Other clothing which may be coated includes undergarments, socks, athletic apparel, surgical apparel, healthcare apparel, shoes and boots.

Other substrates suitable for coating include filters, for example medical filters (including respirator filtration media and fluid filtration media), and other filters including HVAC filtration media, water filtration media and fluid filtration media.

Further suitable substrates include currency, debit/credit cards, industrial waste and water handling equipment, petrochemical and crude oil production, distribution and storage equipment and infrastructure. Additional suitable substrates include personal protective equipment and military apparatus such as face masks, respirators, decontamination suits and gloves.

### EXAMPLES

All chemicals used in this work were obtained from Sigma-Aldrich, Germany, and not purified further. All solvents were HPLC or analytical grade, obtained from Fisher scientific, UK. Gold wire (purity 99.99%) was obtained from Advent research materials, UK. LaSFN9 glass required for SPR measurements was obtained from Berliner glass, Germany. All growth media were sourced from Fisher scientific, UK and were of microbiological grade.

### Example 1A

### Synthesis of PSSM (Phosphine Stabilised Silver Maleimide)

To a stirred solution of silver nitrate (5 mmol, 0.85 g) in ethanol (10 ml), 40 ml of a methanolic solution of triphenylphosphine (10 mmol, 2.62 g) was added, and the solution stirred for 1 hour. In a separate vessel sodium ethoxide (5 mmol, 0.34 g) was added to a stirred methanol solution (10 ml) of maleimide (5 mmol, 0.49 g). Addition of the sodium ethoxide/maleimide solution to the silver nitrate solution resulted in the formation of a clear colourless solution, which was stirred at room temperature for a further 2 hours and then excess solvent removed under vacuum. The resulting solid residue was isolated by filtration and the off-white precipitate collected, washed successively with distilled water (15 ml), methanol (5 ml) and diethyl ether (10 ml) and dried in-vacuo. Recrystalisation from isopropanol results in the formation of crystals suitable for single crystal X-ray diffraction studies.

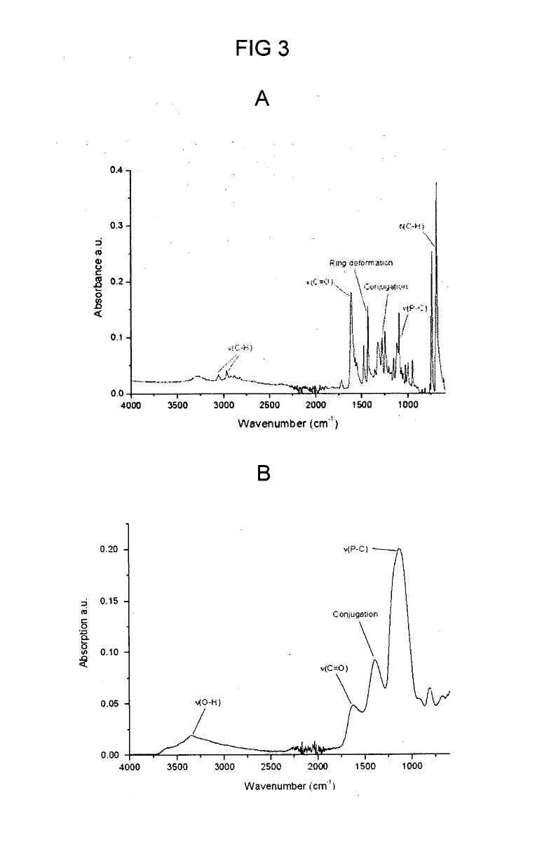

The structure of PSSM is given above. FIG. 3A shows an FT-IR spectrum of PSSM, showing an N—Ag stretch.

### Example 1B

### Synthesis of PTSM (tris-triethylphosphito silver maleimide

To a stirred solution of maleimide (20 mmol, 1.96 g) in ethanol (20 ml), a 10 ml solution of silver nitrate (20 mmol, 3.40 g) in an ethanol/acetonitrile mixture (minimum MeCN) was added and the solution stirred for 5 minutes. After this time a solution of triethylamine (20 mmol, 2.02 g) in ethanol (5 ml) was added dropwise over 30 minutes, resulting in the formation of an insoluble white precipitate. The solid residue was isolated by filtration, washed three times with ethanol and dried in-vacuo, yielding silver maleimide (2.84 g, yield 94%).

To a stirred solution of silver maleimide (10 mmol, 2.04 g) in THF (30 ml), a stirred solution of triethylphosphite (30 mmol, 4.98 g) in THF was added and the solution stirred for 30 minutes. After this time solvent was removed under vacuum, yielding a yellow liquid, tris-triethylphosphito silver maleimide (PTSM) (6.16 g, yield 88%).

PTSM was soluble in deuterated chloroform for NMR study with a very clean spectrum observed.

¹H NMR (300 MHz) (ppm): δ=6.4 (s, 2H, —CH—CH—), 3.9 (p, 18H, P(O—CH₂—CH₃)₃), 1.2 (t, 27H, P(O—CH₂—CH₃). ¹³C NMR (75 MHz) δ=16.2, 59.7, 136.0, 186.2.

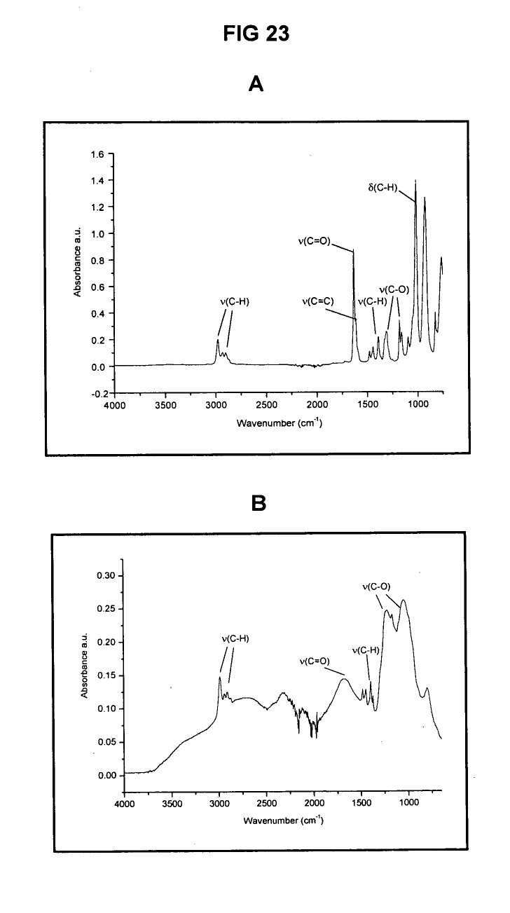

The Structure of PTSM is Given Above. FIG. 23A Shows a FTIR Spectrum of PTSM.

### Example 2A

### Plasma Deposition of PSSM

A reactor comprising a 30 cm-long, 10 cm-diameter Pyrex tube and stainless steel electrodes at each end, attached to an Edwards RV5 pump (used to create a vacuum of ca. 10⁻² mbar) was used for these experiments based on the design described by Bullett et al¹⁷. Oxygen for reactor cleaning was fed in via a gas control valve and monomer vapour introduced via a Young's tap. A 13.56 MHz RF coaxial system supplied power and was connected to the reactor via a 2 mm-diameter coil of copper wire, earthed through the electrodes. A manual matching unit was used to adjust input impedance, ensuring minimal reflected power (>0.5%) and an oscilloscope allowed pulsing of the input power.

Fresh monomer was degassed by subjecting several freeze-thaw cycles in liquid nitrogen under low pressure prior to polymerization. An oxygen plasma was used to clean the reactor for 20 min at 50 W continuous wave input power. The monomer flow rate was determined by opening the monomer chamber to the reactor, then isolating the reactor from the vacuum. The change in pressure over 30 seconds allowed monomer flow rate to be calculated. All plasma polymerization reactions were run with a flow rates of between 2-10 cm³ₛₜₚmin⁻¹, at 50 W input power for three hours. Glass, gold-coated glass (for FT-Infra-red), polystyrene Petri dishes and polypropylene non-woven fabric (from disposable nappies) were used as substrates. Substrates were loaded to the reactor at optimized deposition points each time, with FTIR of the product taken immediately after reaction to ensure reproducibility of the resulting polymer. The mass spectrum of the polymer was obtained by washing the coated glass substrate with hot methanol, with the resultant methanol-polymer solution being analyzed. Gold surfaces were modified to enhance adhesion properties of the polymer to gold, by immersion in 5 mmol dm⁻³ alkyl-mercaptan in ethanol for, 30 mins to form a self-assembled monolayer.

The FT-IR spectrum for the PSSM coating is shown in FIG. 33. IR spectra of monomers and plasma deposited coatings were obtained using a Perkin-Elmer Spectrum 100 FT-IR spectrometer with a diamond/ZnSe ATR accessory. Plasma coatings were formed on gold coated glass discs (Ecochemie NL) and then placed on the ATR crystal, with a coating thickness of approximately 50 nm. This method significantly enhanced measurement sensitivity.

Film thickness was estimated by Surface Plasmon Resonance (SPR). A polymer-coated gold slide was placed on a high refractive index glass prism and the reflectance of P polarized laser light at 633 nm measured as a function of incident light angle. At the angle of surface plasmon resonance, no light is reflected and a minimum observed. The angle of resonance is proportional to the refractive index properties of the polymer film (n.d). The resultant angle spectrum was fitted to a four layer Fresnel model using, with the optical properties of the gold and prism having been obtained previously. By estimating the refractive index of the film it was possible to obtain an approximate value of film thickness. Film thickness was estimated to be 20 nm+/−4 nm following deposition for 1 hour under continuous wave 100 W rf power.

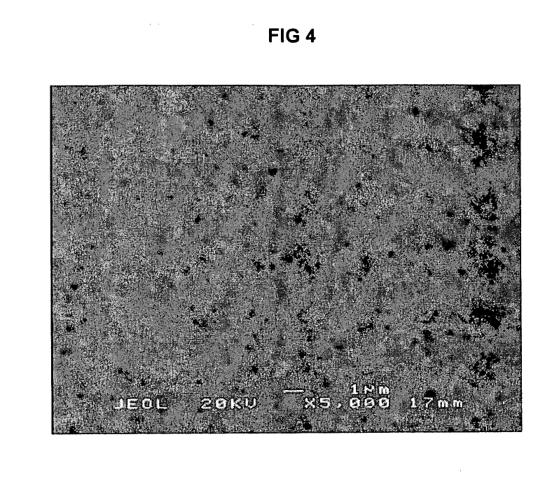

A scanning electron microscope (SEM) image of a PSSM coating on a thin gold film, taken with a JEOL JSM6480LV SEM instrument is shown in FIG. 4. The polymer appears to be morphologically similar to the underlying gold film. The array of dark spots of dimensions between 100-400 nm are believed to be silver particles formed during the plasma deposition process. Direct measurement of swelling by SPR revealed little apparent change in coating thickness following immersion in phosphate buffered saline, suggesting little swelling.

Atomic absorption of PSSM polymer was obtained from a film deposited on oxygen plasma-treated petri dish. The film was deposited for 2 hours at 50 W under continuous wave conditions and soaked in 2 mL phosphate buffered saline solution for 24 hours. Leaching of silver into the buffer solution at a concentration of 0.07 ppm was measured.

### Example 2B

### Plasma Deposition of PTSM

Plasma deposition of PTSM was run at 50 W under 1/40 ms pulse cycle, by an analogous method to that described for PSSM.

A strong correlation between monomer and plasma film structure was seen in using IR. FIG. 23A shows the FTIR spectrum for PTSM and FIG. 23B shows the FTIR spectrum plasma polymerised PTSM. Key absorbances assigned to alkanes were observed as sharp peaks identical to the monomer, while C═O and C—O bands were broadened by oxidation of the film. Removal of strong vinyl C—H bend and C═C stretch indicate elimination of alkene functionality consistent with polymerisation through the maleimide unit. The strength of film absorbances outweighed contribution of O—H band appearance due to oxidation, indicating a relatively thick film was deposited which was believed to be related to high flow rate of monomer.

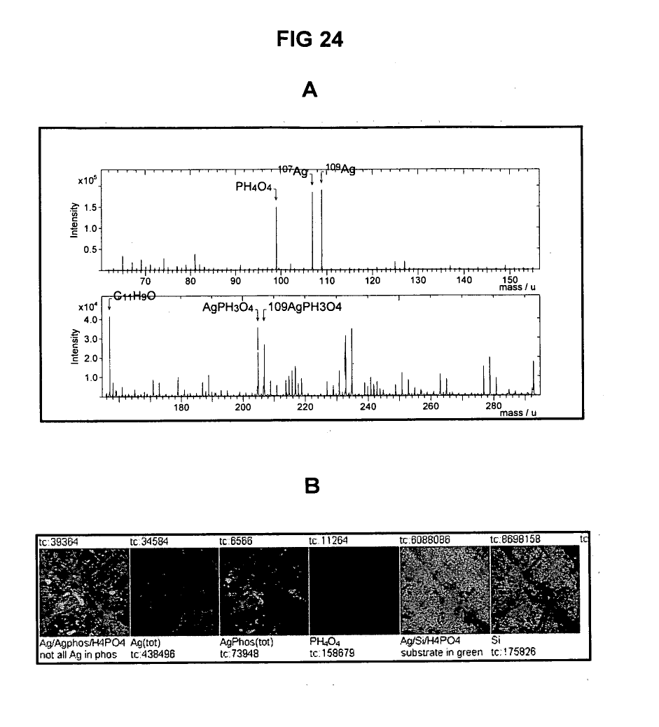

SIMS was used to map silver content on the surface of plasma polymerised PTSM on silicon wafers. The SIMS spectrum and SIMS maps are shown in FIG. 24. Peaks assigned to pure silver ions and phosphate-silver moieties were seen and interestingly no phosphite bound silver was detected in major quantities. It could be the case that ion impact on the surface caused cleavage of these bonds, or they were broken by ionisation in the plasma reactor. In either scenario, silver was observed inhomogenously in large quantities across the surface and generally accompanied by organic constituents, indicating potential for a surface which is highly antimicrobial. The antimicrobial properties of the film are demonstrated below.

### Example 3

### Microbiology of PSSM and PTSM

### Example 3A

FIG. 5 shows the zone of inhibition around pellets of PSSM monomer for S. aureus (A), M. luteus (B) and P. aeruginosa (C), after 24 h at 37° C. FIGS. 5A and B show a sharp ring of inhibition around the central light-coloured pellet. FIG. 5C shows more blurred inhibition zones of a light colour around the pellets.

### Example 3B

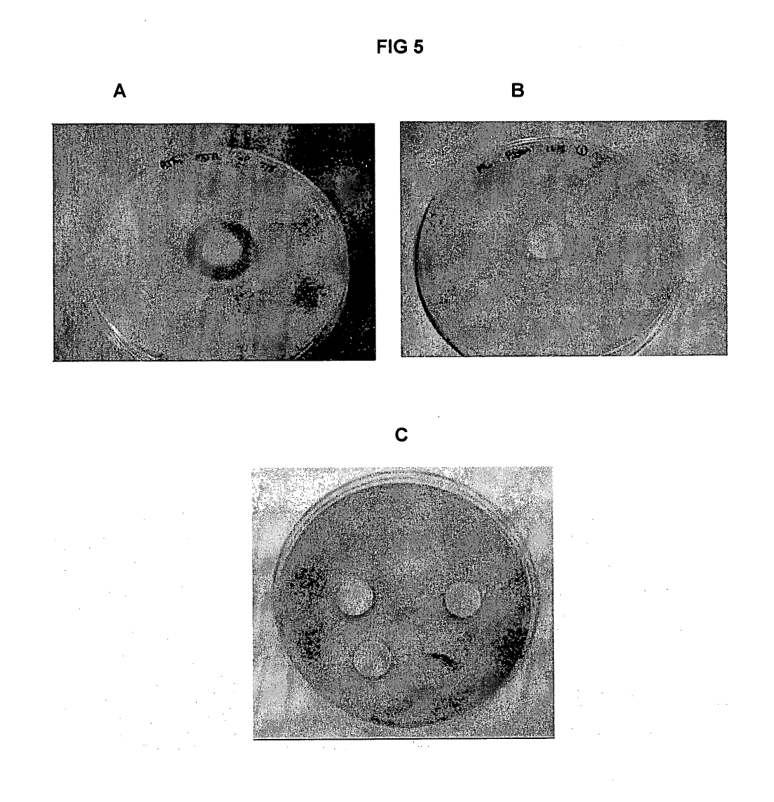

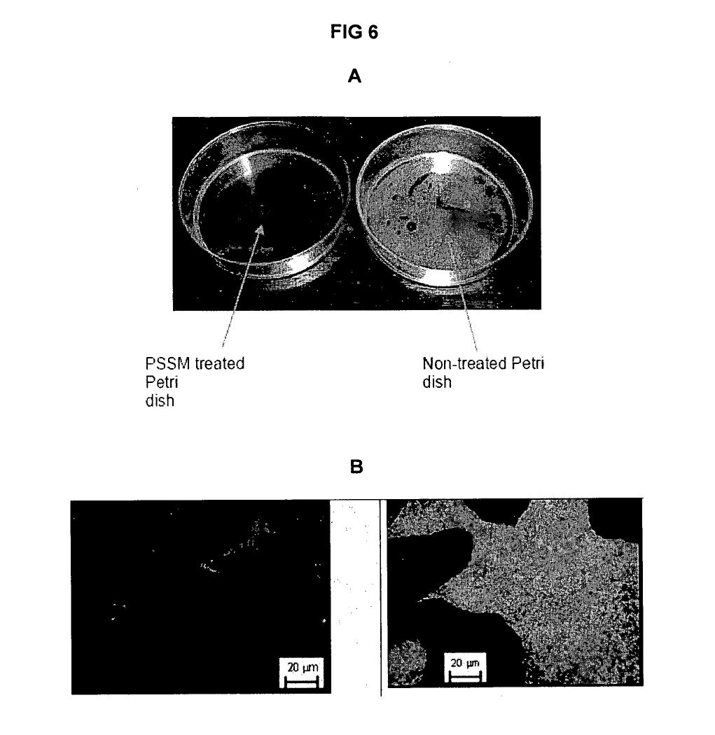

Polystyrene Petri dishes were coated with a plasma deposited PSSM coating. Colonisation by M. luteus after 24 hours growth in LB media was observed, with the results shown in FIG. 6A. The dish on the left (coated dish) shows little M. luteus growth, in contrast to the untreated dish on the right.

An assay for measurement of bacterial cell viability was performed to establish how bacteria were affected by the PSSM polymer film, i.e. did the film kill surface attached bacteria? In this experiment, polystyrene Petri dishes were coated by plasma deposition of PSSM. The assay, purchased from Invitrogen, uses two dyes: a nucleic acid dye, propidium iodide which is excited at 470 nm and has a emission maximum at 630 nm (red). Propidium iodide only enters at a significant concentration into cells that are dead/dying. Hence dead bacteria appear red when excited by 470 nm light. Live cells are observed by a fluoresceine derivative (SYTO 9). SYTO9 is also excited at 470 nm but has an emission maximum at 540 nm (green) enters all cells, and is used at a lower concentration than the propidium iodide.

FIG. 6B shows the results. Dead bacteria fluoresce red (shown as a dim fluorescence in FIG. 6B, image on the left). Live bacteria fluoresce green (shown as a brighter fluorescence in FIG. 6B, image on the right). It can clearly be seen that in the image on the left (treated Petri dish), only isolated clusters of dead bacteria are seen. In contrast, on the untreated dish thick mats of live bacteria are seen, with evidence of colony formation.

### Example 3C

A study of S. aureus (MSSA) growth on Petri dishes coated with a plasma deposited PSSM coating, using a BAC-light Live-Dead stain was performed.

Bacteria were grown for 24 hours at 37° C. on a Petri dish which was pre-treated with oxygen plasma and PSSM polymer deposited for 2 hours at 50 W under continuous wave conditions. Control Petri dishes used were oxygen plasma-treated. After incubation effluent media was aspirated, replaced with fresh media which was removed and left to dry for 10 minutes. 50 μL of BAC-Light LIVE/DEAD 2× stock solution was added to the surface and incubated in the dark for 15 minutes. After this time the surface was washed with fresh media and allowed to dry. This yielded a surface suitable for fluorescence microscopy.

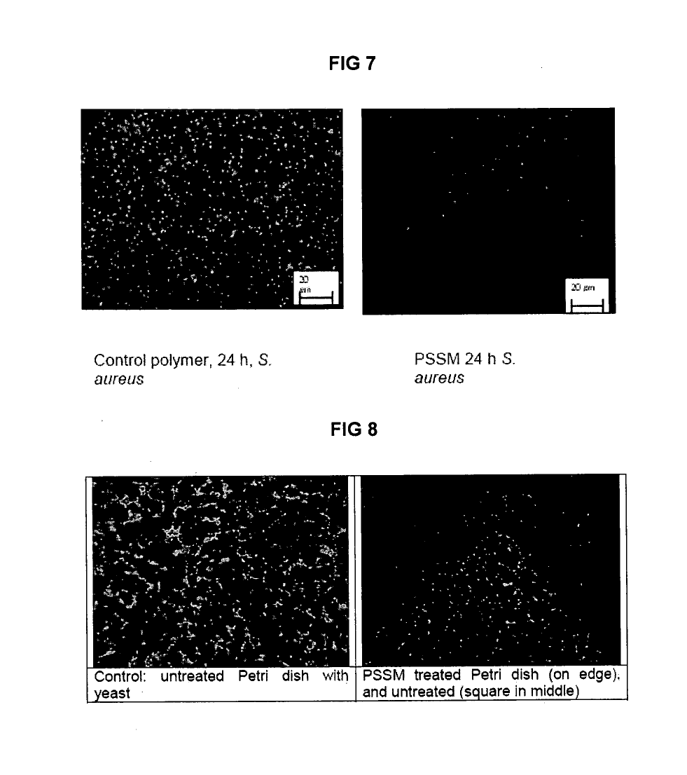

Live bacteria fluoresce green (shown as bright fluorescence in FIG. 7, particularly the left-hand image) and dead bacteria fluoresce red (shown as duller fluorescence in FIG. 7, particularly the right-hand image). The results are shown in FIG. 7. The left-hand image shows S. aureus on an uncoated Petri dish after 24 h, and the right-hand image shows S. aureus on a Petri dish with PSSM a plasma deposited coating after 24 h. The results show far fewer bacteria on the treated Petri dish, and that almost all bacteria which are present are dead.

### Example 3D

The effect of PSSM plasma deposited coatings on S. cerevisiae was studied. The results are shown in FIG. 8. The image on the left shows an untreated Petri dish. The image on the right shows a patterned Petri dish with a PSSM plasma deposited coating at the edge, and an untreated triangle in the middle. FIG. 8 clearly shows that the growth of yeast in inhibited by the PSSM coating, but that yeast grows well on the untreated surface.

### Example 3E

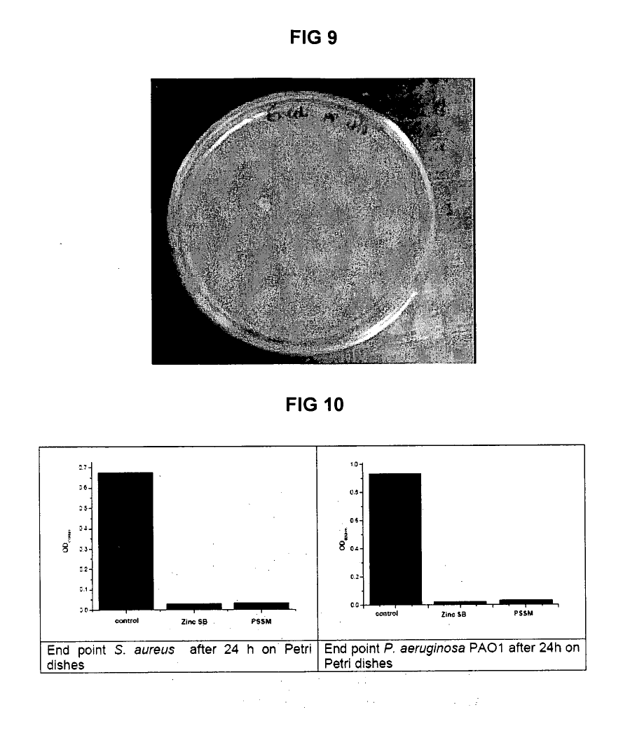

PSSM coatings were plasma deposited on squares of polypropylene non-woven. E. coli were grown on treated and untreated squares of polypropylene non-woven on LB broth/agar gels. The results are shown in FIG. 9. The two treated squares are clearly seen on the left-hand side, clear of E. coli. The untreated squares are not clearly visible, and show E. coli growth.

### Example 3F

FIG. 10 shows end point measurements after 24 hours for both S. aureus and P. aeruginosa on untreated, PSSM coated and ZSB coated Petri dishes. 5 mL suspensions of each bacteria in LB broth were grown for 24 hours at 37° C. in the Petri dishes. The initial bacterial concentration at t=0 was 200 cfu/mL. After 24 hours the suspension was removed and the optical density quantified at 600 nm in a UV/Vis spectrometer.

### Example 3G

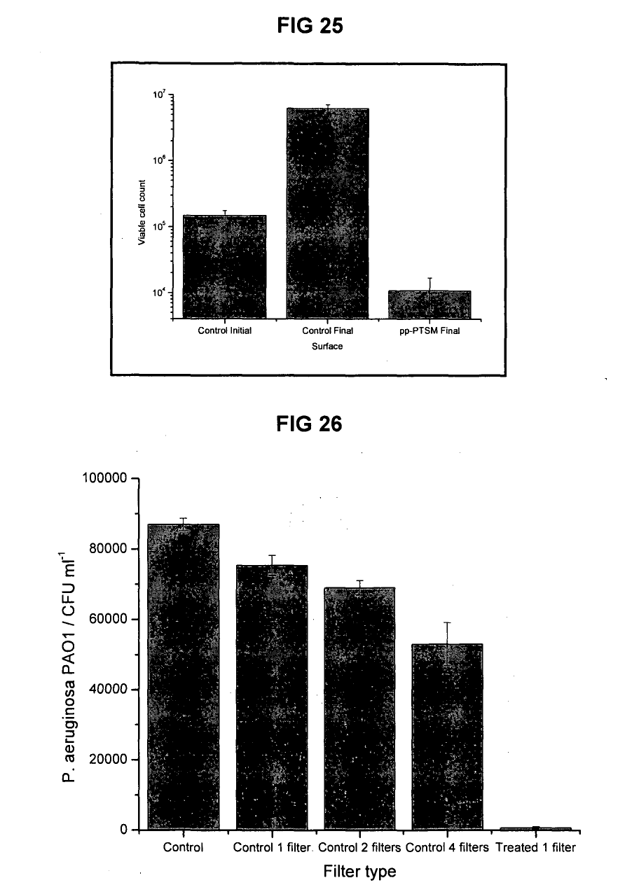

A single assay was conducted on a plasma polymerised film of PTSM with a fluorinated adlayer on non-woven against Pseudomonas aeruginosa. The results are shown in FIG. 25. The result was dramatic, with a huge difference in cell viability between those taken from the treated and non-treated squares of material (around 99.9% reduction in cell viability). The film decreased the number of cells from those initially inoculated so showing outright killing of cells on the surface, not just growth inhibition.

### Example 4A

### Synthesis and Deposition of Zinc Schiff-Base Complex (ZSB)

An example synthesis of ZSB is illustrated in FIG. 2, as “Ligand System 1”, and as illustrated below:

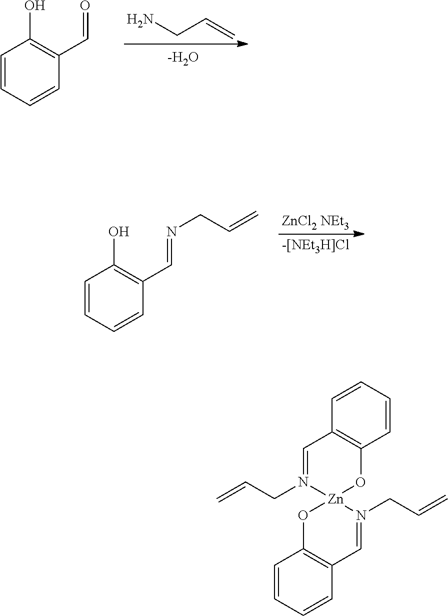

The synthesis of the Schiff-base ligand used in this metal ion complex was carried out as described below.

N-n-Propenylsalicylaldimine (Ligand 1). To a freshly prepared MeOH (40 ml) solution of salicyaldehyde (15.20 ml, 100 mmol) was added allylamine (7.40 ml, 100 mmol). The mixture was heated and allowed to cool to room temperature and left for 60 minutes, at which point a yellow solution was produced, the solvent was removed via rotary evaporation. 50 ml of Dichloromethane (DCM) is added to the resulting oil and the solution is dried using Magnesium Sulphate. MgSO₄ is then removed via gravity filtration and DCM is removed by rotary evaporation to afford ligand 1 (12.692 g or 78 mmol, a yield of 78%).

Complexation. To a MeOH solution (40 ml) of Ligand 1 (8.05 g, 50 mmol) was added Zinc chloride (ZnCl₂) (3.40 g, 25 mmol) and triethlyamine (8.50 ml, 50 mmol). The reaction was refluxed for two hours and allowed to cool to room temperature. At which point the solvent was removed under vacuum, a yellow solid precipitated out of the resulting oil after scratching with a glass rod. The resulting solid was dissolved in 50 ml DCM, and aqueous impurities were removed via a water wash. The resulting organic layer was dried using MgSO₄, and the solvent was removed under vacuum again. The product was recrystallised in hot toluene to afford complex 1 (7.82 g) as a yellow powder. Melting temperature 112-114° C.

ZSB coatings can be deposited by using an analogous plasma deposition method to that described above for PSSM.

### Example 4B

### Synthesis and Deposition of Copper Schiff-Base Complex (CSB)

The synthesis of the CSB complex was identical to that of the ZSB complex, except that copper acetate (25 mmol, 4.54 g) is added in place of the zinc chloride. The work up was analogous to that for the ZSB complex. The product was a dark green crystalline solid. The product was recrystallised in the minimum volume of hot toluene to afford a dark green crystalline solid.

Yield 69%

M.p 120° C.

CSB coatings can be deposited by using an analogous plasma deposition method to that described above for PSSM.

### Example 4C

### Optimisation of Plasma Deposition Parameters

In order to determine the optimum plasma deposition conditions in terms of pulse sequence, a series of CSB coatings were made onto polystyrene Petri dishes (with no fluorination) and the relative degree of bacteria killing/growth suppression measured using a bacteria live-dead fluorescent assay (Invitrogen). This assay was used to stain live bacteria green, and dead red. After removal from the surface by vortexing, the live-dead ratio was determined by fluorescence emission ratios.

The deposition parameters were:

| | | | Flow | | | |
|---|---|---|---|---|---|---|
| | Duty Cycles | Monomer | rate/ | Base | Film | Deposi- |
| Com- | (peak | temper- | sscm³ | pressure/ | depos. | tion |
| pound | power) | ature | min⁻¹ | mbar | rate | time |
| CSB | CW, 40/40, | 175° C. | 11 | 0.022 | 1 nm | 30 min |
| | 10/40 1/40 | | | | min⁻¹ | |
| | (50 W) | | | | | |

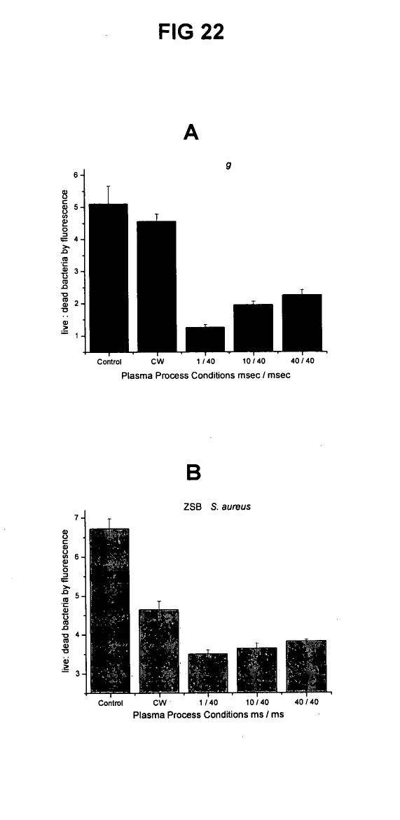

The results are shown in FIG. 22. They suggest that the optimum plasma conditions are pulsed, with a cycle around 1 millisecond on/40 millisecond off (1/40). This is believed to be due to relatively greater retention of the Schiff base ligand in the film under these low power conditions.

(In FIG. 22, CW means continuous wave plasma deposition.)

### Example 4D

### Plasma Deposited Film Analysis

Mass of the deposited films was estimated by depositing compounds onto quartz crystal microbalance chips and measuring the frequency shift, and estimating the mass change using the Sauerbrey equation¹⁸. Film thickness was measured using a TLC Tencor P-10 alpha step profiler.

(i) Film Properties—30 min deposition, 1/40 ms (1 ms on, 40 ms off) duty cycle

| | pp-ZSB | pp-CSB |
|---|---|---|
| Mass (μg cm⁻²) | 14.57 | 6.42 |
| Moles of monomer | 18.87 | 16.67 |
| (nmol cm⁻²) | | |
| Thickness (nm) | 32 +/− 2 | 30 +/− 4 |
| Density (g/cm³) | 4.55 | 2.14 |

### (ii) Transmission Electron Microscopy

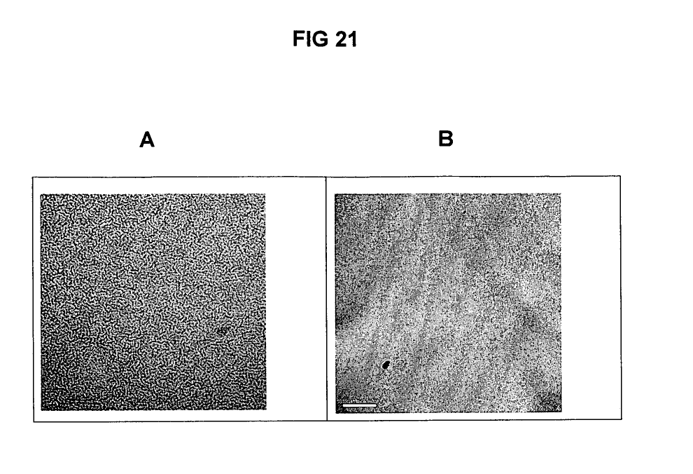

Pulse plasma deposited pp-ZSB and pp-CSB films were formed on copper TEM grids and studied by Transmission Electron Microscopy (TEM) on a JEOL JEM1200 microscope. The pp-ZSB shows a microstructure repeat pattern, while the pp-CSB film appears to be considerably smoother. The images are shown in FIG. 21.

(iii) Secondary Ion Mass Spectrometry (SIMS)

SIMS measurements were made on instruments at the National Physical Laboratory, London. The SIMS instrument was an ION-TOF spectrometer, using a bismuth ion beam for impacting the surface. The SIMS software also allowed imaging of the surface, looking at variation in surface density of various ion fragements.

SIMS measurements demonstrated the presence of zinc in the ZSB films, identifying ⁶⁴Zn at 63.9 mass units in the positive ion spectrum. SIMS imaging of the surface suggested that no specific macrostructure relating to zinc was observed.

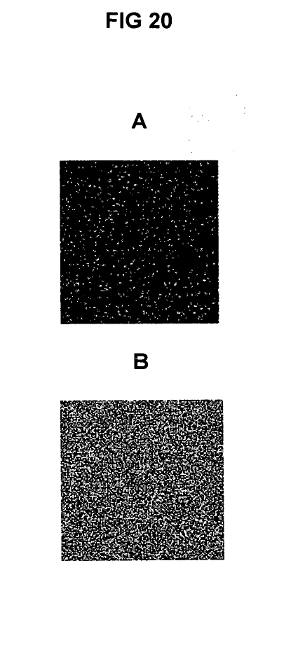

FIG. 20A shows the SIMS map of ⁶⁴Zn on the surface. The image dimensions are 500×500 mm. The zinc, although at low levels, appears to be distributed across the surface as particles.

FIG. 20B shows the SIMS map of organic fragment C₃H₁₆N⁺.

### Example 5

### Microbiology of ZSB

### Example 5A

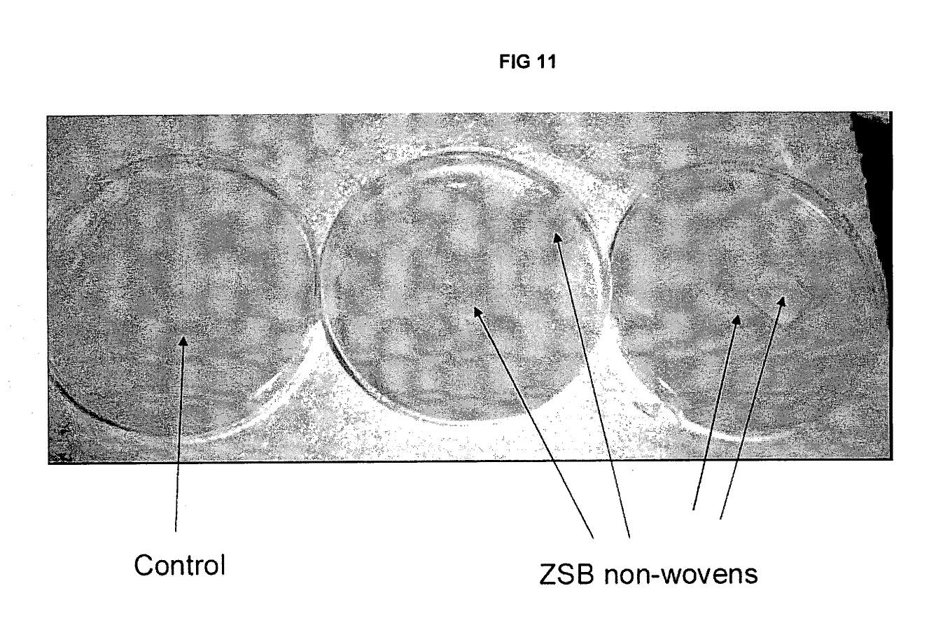

Non-woven polypropylene top sheet was removed from Boot™ nappies and plasma deposited with ZSB, following the protocol given above in Example 2. Squares of coated non-woven are shown in FIG. 11, in the two right-hand culture plates. The control (without any non-woven) is on the left. S. aureus was cultured on the plates for 24 hours. Growth on the two right-hand plates is suppressed due to the presence of the ZSB coated non-wovens, which were moved at the end of the experiment to expose a square with little or no S. aureus growth.

### Example 5B

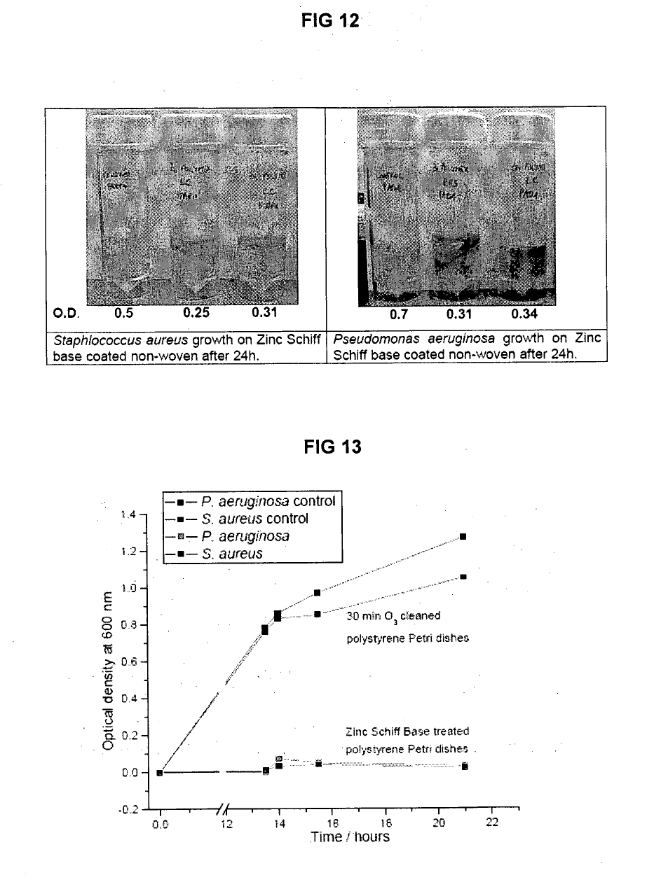

A study of the antimicrobial action of the ZSB coated non-wovens was obtained by growing S. aureus and P. aeruginosa in ZSB non-woven (1×1 cm) and measuring the optical density of the bacteria after incubation of bacteria and non-woven in LB media for 24 hours at 37° C. The results are shown in FIG. 12, with the left-hand sample of each set of three being the control (oxygen plasma treated non-woven). The O.D. numbers are the optical densities of bacteria at 600 nm.

### Example 5C

A time course measurement of bacterial growth (measured by optical density at 600 nm) was performed on ZSB coated Petri dishes. The results are given in FIG. 13, and show almost complete inhibition of growth over 24 hours.

### Example 5D

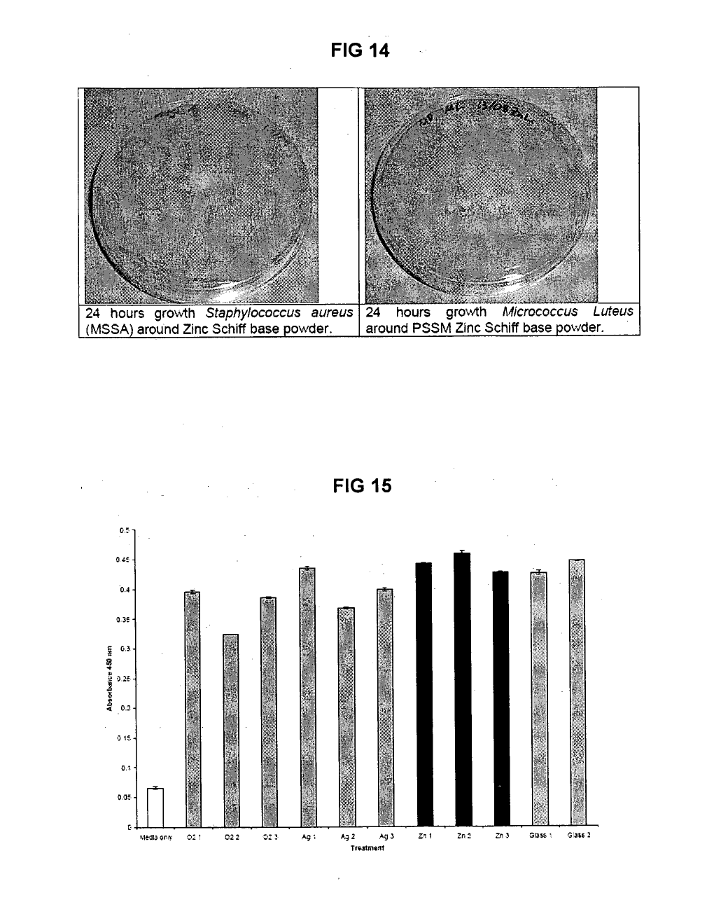

ZSB powder was used to show antimicrobial efficacy. Zones of inhibition around the ZSB powder are visible for both S. aureus and M. luteus after 24 hours and 37° C., and are shown in FIG. 14.

### Example 5E

Minimum Inhibition Concentration Determination for ZSB and CSB.

An initial 6 solution dilution series of ZSB and of CSB was made up in microbiology grade DMSO (Sigma, >99.99% purity) with a range of 20 mmol dm⁻³ to 0.325 mmol dm⁻³. DMSO was used due to the insoluble nature of the two Schiff base complexes. Once this initial dilution series was made up, 100 μl of each solution was added to a 900 μl bacterial solution (CFU 10²). This dilutes the solutions by a factor of ten to give final concentrations of 2, 1, 0.5, 0.25, 0.125, 0.0625 and 0.01325 mmol dm⁻³ complex solution in a 1 ml bacterial solution (approx CFU 10²). The samples were incubated at 37° C. for 18 hours, after which they were removed from incubation and the optical density determined 600 nm measured. S. aureus MSSA 476 and P. aeruginosa PAO1 were used to asses the antimicrobial activity of the monomers, and later the plasma deposited films. The results of this assay provided an estimate for the minimum concentration required to inhibit growth of both bacteria.

The ability of ZSB and CSB to kill S. aureus and P. aeruginosa was quantified using standard Minimum Inhibition Concentration assays. The results are summarized in the table below.

### MIC Results

| | S. aureus MSSA 476 | | P. aeruginosa PA01 | |
|---|---|---|---|---|
| Complex | IC50 (μM) | IC50 (μg/ml) | IC50 (μM) | IC50 (μg/ml) |
| ZSB | 270 | 208 | 320 | 415 |
| CSB | 500 | | >1000 | |

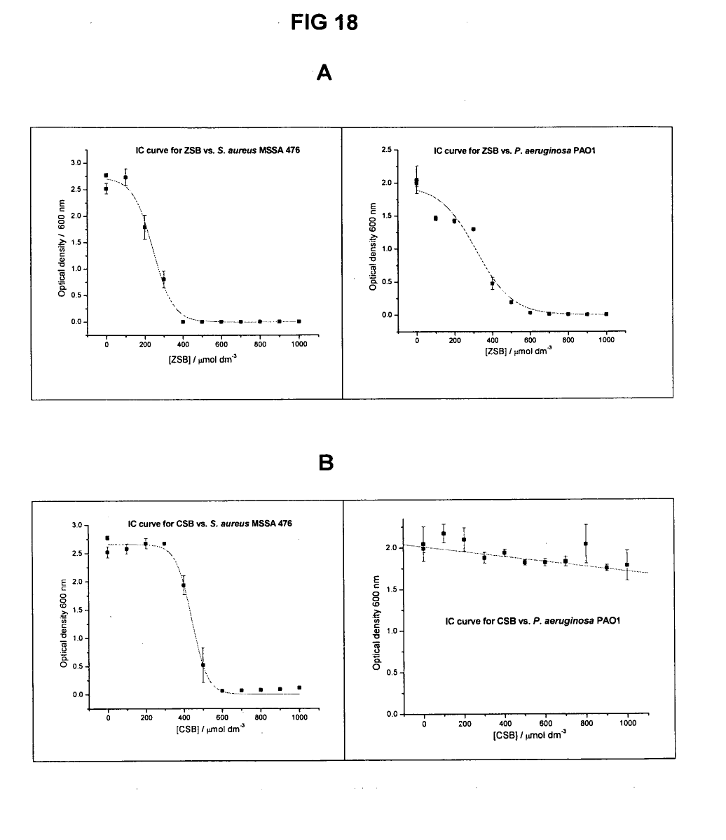

The MIC curves (shown in FIG. 18) show that the CSB complex had no measureable inhibition of P. aeruginosa, though was effective against S. aureus.

### Example 5F

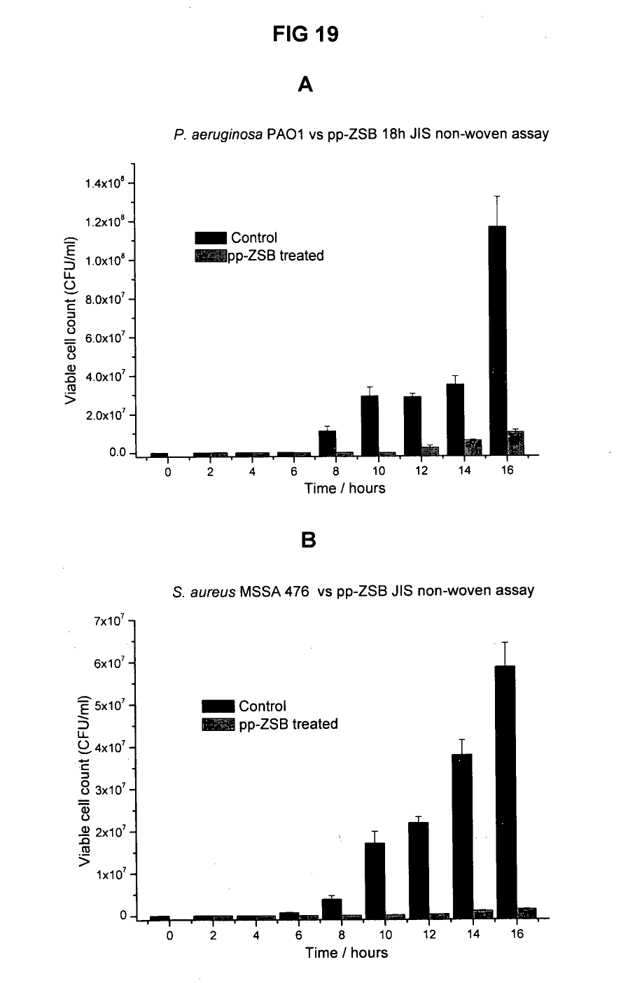

ZSB was coated under 1/40 pulse conditions onto 1 cm² of non-woven polypropylene (obtained from the topsheet of Boots™ disposable nappies (diapers). Following ZSB deposition, the fabric was treated with a 30 s C₂F₆ plasma to render the surface more hydrophobic. All fabric squares (and controls) were measured in triplicate. The non-woven fabric was inoculated with 200 μl of a suspension of either the P. aeruginosa or S. aureus in LB broth at a concentration of 10⁵ cfu ml⁻¹ (vide-supra). Every two hours, for a maximum of 16 hours, fabric squares were removed and the bacterial concentration on the fabric quantified by colony counting. Results are shown below in FIGS. 19 A and B.

### Example 5G

ZSB was coated by plasma polymerisation onto a sheet approximately 5 cm×10 cm of 2.5 mm thick Vyon® filter material. The material was then cut into 5 mm disks. The filter discs were placed inside 1 ml disposable pipette tips and P. aeruginosa bacteria suspended in physiological saline applied (1 mL). Gentle pressure was used to push the liquid through the filters, over about 1 minute.

The liquid was passed through control filters with 1, 2, 3 and filter discs, and through a single plasma coated filter disc. The eluted liquid was cultured on nutrient rich agar plates for 18 hours, and colonies counted. The results are shown in FIG. 26. The concentration of P. aeruginosa eluted through the filters is shown. A reduction in bacterial concentration of greater than 10⁴ (4 orders of magnitude) was measured for the coated filter: from 10⁵ to 7 CFU

### Example 6

### Cytotoxicity

NIH 3T3 (Embryonic swiss mouse fibroblasts) cells were plated onto ZSB and PSSM plasma coated coverslips at high density and allowed to settle and grow for 24 h. Controls included cells seeded onto plain glass coverslips as well as the oxygen plasma-treated slides—in case there was any cytotoxicity resulting from this coating. Three coverslips were used for each plasma coating. The results are shown in FIG. 15, with ZSB marked “Zn” and PSSM marked “Ag”.

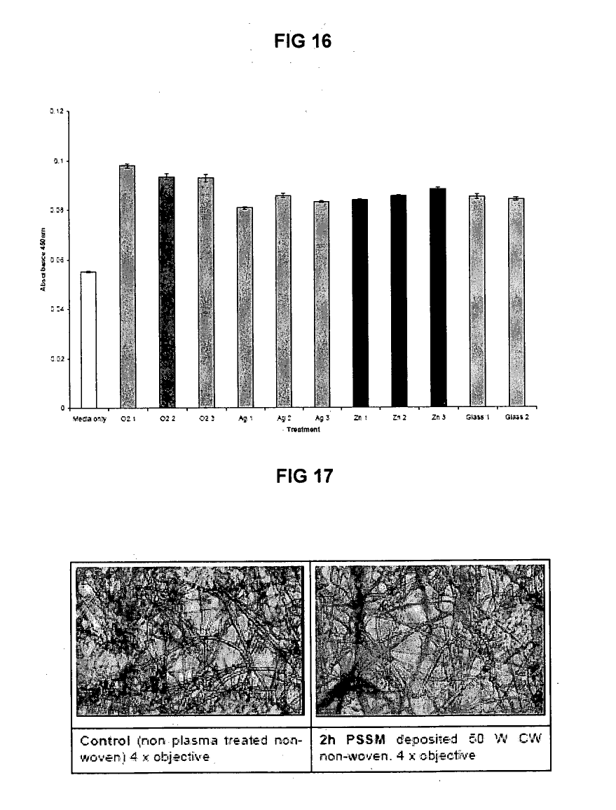

The treated glass coverslips, plus controls were also cultured with human neonatal epidermal cells. These EK102-05n (Human Neonatal Epidermal Keratinocytes) cells are much slower growing and were only available for use at a lower density. Therefore cells were allowed to grow on the treated coverslips for 72 h prior to cytotoxicity testing. Three coverslips for each coating were used as with the NIH 3T3 cells, and the results are shown in FIG. 16.

### Example 7

### Effect of Deposition Process on Non-Wovens

PSSM was deposited onto non-woven top sheet, taken from disposable nappies. The non-woven was examined by light microscopy before and after deposition to check for signs of structural change in the polypropylene. No change was seen, as demonstrated by the images in FIG. 17.

### Example 8

### Fluorination of ZSB and CSB Films

The pp-ZSB and pp-CSB films were both very hydrophilic on removal from the reactor. X-ray Photoelectron Spectroscopy analysis suggested a high degree of oxidation of the film, possibly post-treatment. In order to improve the antimicrobial efficacy of fabrics coated in ZSB or CSB, the films were exposed to a brief (30 second) plasma of hexafluoroethane (C₂F₆) immediately following plasma deposition of the organometallics. This made the coated fabrics extremely hydrophobic (water droplets formed spheres and ran off) and further improved antimicrobial performance.

### REFERENCES

1. E. Paintsil, Current Opinion in Pediatrics, 2007, 19, 75-82.
2. M. I. Gomez, A. Prince, Current Opinion in Pharmacology, 2007, 7, 244-251.
3. J. W. Costerton, P. S. Stewart, E. P. Greenberg, Science, 1999, 284, 1318-1322
4. H. Ceri, M. E. Olson, C. Stremick, R. R. Read, D. Morck, A. Buret, Journal of Clinical Microbiology, 1999, 37, 1771-1776.
5. N. Kurosaka, T. Nihei, H. Kumada, T. Umemoto, Y. Kondo, N. Yoshino, T. Teranaka, Journal of Dental Research, 2000, 79, 2569-2574
6. N. P. Desai, S. F. A. Hossainy, J. A. Hubbell, Biomaterials, 1992, 13, 417-420.
7. P. Favia, R. d'Agostino, Procedes Plasma Froids, 2003, 303, 40-48.
8. [M. Allmyr, F. Harden, L.-M. L. Toms, J. F. Mueller, M. S. McLachlan, M. Adolfsson-Erici, G. S.-Englund, Science of the Total Environment, 2008, 393, 162-167.
9. J. Gil-Toma's, S. Tubby, I. P. Parkin, N. Narband, L. Dekker, S. P. Nair, M. Wilson, C. Street, Journal of Materials Chemistry, 2007, 17, 3739-3746.
10. G. J. Gabriel, A. Som, A. E. Madkour, T. Eren, G. N. Tew, Materials Science and Engineering R, 2007, 57, 28-64.
11. K. I. Batarseh, Journal of Antimicrobial Chemotherapy, 2004, 54, 546-548.
12. J. M. Schierholz, L. J. Lucas, A. Rump, G. Pulverer, Journal of Hospital Infection, 1998, 40, 257-262.
13. S. Silver. FEMS Microbiology Reviews 2003, 27, 341-353.
14. D. Lee, R. E. Cohen, M. F. Rubner, Langmuir, 2005, 21, 9651-9659.
15. A. Brooun, S. Liu, K. Lewis, Antimicrobial Agents in Chemotherapy, 2000, 44, 640-646.
16. D. Hegemann, M. Mokbul Hossain, E. Komer, D. J. Balazs, “Multifunctional surfaces through nanostructured plasma coatings” Conference Information: 5th International Nanotechnology Symposium, NOV 21-22, 2006 Karlsruhe, GERMANY Source: Nanofair 2006 New Ideas for Industry Book Series: VDI BERICHTE Volume: 1940 Pages: 37-40 Published: 2006
17. N. A. Bullett, J. D. Whittle, R. D. Short, C. W. Ian Douglas J. Mater. Chem., 2003, 13, 1546-1553.
18. Mecea, V. M. Sensors and Actuators A, 1994, 40 1-27.

1. A method of forming an antimicrobial coating on a surface of a substrate, comprising exposing a surface of the substrate and a plurality of metal ion complexes to a plasma environment, each metal ion complex comprising a metal ion having a plasma polymerisable ligand coordinated thereto, and thereby causing plasma deposition of an antimicrobial coating on the surface of the substrate by plasma polymerisation.

2. A method according to claim 1 wherein the polymerisable ligand comprises a plasma polymerisable moiety, for example a C—C double bond or a C—C triple bond.

3. A method according to claim 1 wherein the ligand comprises a coordination moiety.

4. A method according to claim 3 wherein the coordination moiety includes a moiety selected from acyl, imide, amine, carbonyl, dicarbonyl, cyanyl, nitro, a Schiff-base and hydroxyl.

5. A method according to claim 1 wherein the ligand is maleimide optionally substituted by up to two substituents, or a ligand having general formula III: 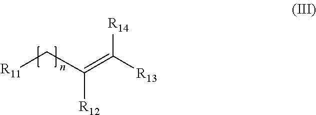
wherein
n is an integer from 0 to 10, preferably from 0 to 5, more preferably from 0 to 2 or 0 to 1;
one or more of the CH₂ groups within [ ]ₙ may optionally be replaced by Si(R₁₅)₂, wherein each R₁₅ is independently H or saturated or unsaturated, substituted or unsubstituted C₁₋₁₀ alkyl;
R₁₁ is a coordination moiety suitable for coordinating the ligand to the metal ion, preferably including a moiety selected from acyl, imide, amine, carbonyl, dicarbonyl, cyanyl, nitro, a Schiff-base and hydroxyl; and
each of R₁₂, R₁₃ and R₁₄ is independently selected from H, F, optionally substituted C₁₋₁₀ alkyl, optionally substituted C₆₋₁₀ aryl and optionally substituted C₅₋₁₀ heteroaryl.

6. A method according to claim 1 wherein the ligand is selected from maleimide, acrylate, tiglate, crotonate, cinnamate, the Schiff-base ligands having the structure: 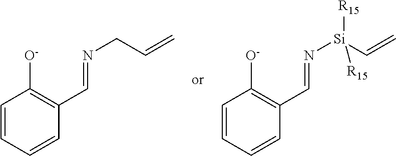
and fluorinated analogues thereof.

7. A method according to claim 1 wherein the metal ion is a Ag, Cu, Zn, Au, Pt or Bi ion.

8. A method according to claim 1 wherein the metal ion complexes include a metal ion complex according to the general formula I, 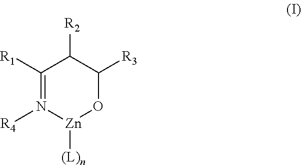
wherein
each R₁, R₂ and R₃ is independently H, substituted or unsubstituted, saturated or unsaturated C₁₋₁₀ alkyl, or R₁ and R₂ or R₂ and R₃ together form substituted or unsubstituted C₆₋₁₀ cycloalkyl, substituted or unsubstituted C₆₋₁₀ aryl or substituted or unsubstituted C₄₋₁₀ heteroaryl;
each R₄ independently is substituted or unsubstituted, saturated or unsaturated C₁₋₁₀ alkyl, which alkyl may include one or more Si atoms in place of C atoms;
provided that at least one of R₁, R₂, R₃ and R₄ has a C—C double bond or a C—C triple bond;
and wherein n is an integer from 1-6, more preferably from 1 to 4 or from 1 to 2, and each L is a further ligand.

9. A method according to claim 8, the metal ion complex having the general formula Ia 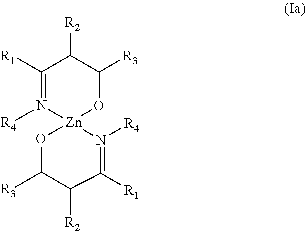
wherein
each R₁, R₂ and R₃ is independently H, substituted or unsubstituted, saturated or unsaturated C₁₋₁₀ alkyl, or R₁ and R₂ or R₂ and R₃ together form substituted or unsubstituted C₆₋₁₀ cycloalkyl, C₆₋₁₀ aryl or C₄₋₁₀ heteroaryl; and
each R₄ independently is substituted or unsubstituted C₁₋₁₀ alkyl having at least one C—C double bond or at least one C—C triple bond, which C₁₋₁₀ alkyl may include one or more Si atoms in place of C atoms.

10. A method according to claim 1 wherein the metal ion complexes include a metal ion complex according to the general formula II 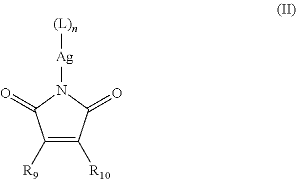
wherein
R₉ and R₁₀ are each independently selected from H and substituted or unsubstituted, saturated or unsaturated C₁₋₁₀ alkyl;
n is an integer from 1-6, more preferably from 1 to 4 or from 1 to 2; and
each L is a further ligand.

11. A method according to claim 10, the metal ion complex having the general formula IIa 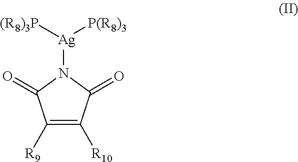
wherein
each R₈ is independently selected from substituted or unsubstituted, saturated or unsaturated C₁₋₁₀ alkyl, C₆₋₁₀ cycloalkyl, C₆₋₁₀ aryl and C₄₋₁₀ heteroaryl; and
R₉ and R₁₀ are each independently selected from H and substituted or unsubstituted, saturated or unsaturated C₁₋₁₀ alkyl.

12. A method according to claim 1 wherein the substrate is selected from an incontinence pad, a nappy, an undergarment, a tampon, a sanitary towel, a wound dressing, a burn dressing, a bandage, a surgical seed, a catheter (such as a urinary catheter, a vascular access catheter, an epidural catheter), a vascular access port, an intravascular sensor, a tracheotomy tube, a percutaneous endoscopic gastrostomy tube, an endotracheal tube, an implantable prosthetic device (such as a stent), a gowns (such as a surgical gown), a surgical mask, a protective glove (such as a surgical glove or an examination glove), a curtain, a uniform, bedding (such as pillow cases, mattress covers and sheets), a surgical drapery, a surgical sock, furniture (such as a table e.g. a bedside table, a bed, and a seating surface), a storage container, a filter, a service tray, a container (such as a food storage container), a conveyor, a blade, a mixer, a roller a kitchen utensil (such as a cutting or serving implement), a food preparation surface, flexible packaging, rigid packaging, a door handle, a sock, athletic apparel, surgical apparel, healthcare apparel, a shoe, a boot, a filter, (for example a medical filter (including respirator filtration media and fluid filtration media) and other filters including HVAC filtration media, water filtration media and fluid filtration media), currency, a debit/credit card, industrial waste and water handling equipment, petrochemical and crude oil production, distribution and storage equipment and infrastructure, and personal protective equipment and military apparatus (such as a face mask, a respirator, a decontamination suit).

13. An antimicrobial coating formed by the method of claim 1.

14. A substrate having an antimicrobial coating formed on a surface thereon, wherein the coating is formed by exposing a surface of the substrate and a plurality of metal ion complexes to a plasma environment, each metal ion complex comprising a metal ion having a plasma polymerisable ligand coordinated thereto, and thereby causing plasma deposition of an antimicrobial coating on the surface of the substrate by plasma polymerisation.

15. A substrate according to claim 14 wherein the substrate is selected from an incontinence pad, a nappy, an undergarment, a tampon, a sanitary towel, a wound dressing, a burn dressing, a bandage, a surgical seed, a catheter (such as a urinary catheter, a vascular access catheter, an epidural catheter), a vascular access port, an intravascular sensor, a tracheotomy tube, a percutaneous endoscopic gastrostomy tube, an endotracheal tube, an implantable prosthetic device (such as a stent), a gowns (such as a surgical gown), a surgical mask, a protective glove (such as a surgical glove or an examination glove), a curtain, a uniform, bedding (such as pillow cases, mattress covers and sheets), a surgical drapery, a surgical sock, furniture (such as a table e.g. a bedside table, a bed, and a seating surface), a storage container, a filter, a service tray, a container (such as a food storage container), a conveyor, a blade, a mixer, a roller a kitchen utensil (such as a cutting or serving implement), a food preparation surface, flexible packaging, rigid packaging, a door handle, a sock, athletic apparel, surgical apparel, healthcare apparel, a shoe, a boot, a filter, (for example a medical filter (including respirator filtration media and fluid filtration media) and other filters including HVAC filtration media, water filtration media and fluid filtration media), currency, a debit/credit card, industrial waste and water handling equipment, petrochemical and crude oil production, distribution and storage equipment and infrastructure, and personal protective equipment and military apparatus (such as a face mask, a respirator, a decontamination suit).

16. A substrate according to claim 14 for use in a method of medical treatment.

17. A method according to claim 2 wherein the ligand comprises a coordination moiety.

18. A method according to claim 17 wherein the coordination moiety includes a moiety selected from acyl, imide, amine, carbonyl, dicarbonyl, cyanyl, nitro, a Schiff-base and hydroxyl.
